(19) United States
(12) Patent Application Publication
(10) Pub. No.: US 2023/0157594 A1
(43) Pub. Date: May 25, 2023

(54) SYSTEMS, DEVICES, AND METHODS FOR PERFORMING FETAL OXIMETRY AND/OR FETAL PULSE OXIMETRY USING A TRANSVAGINAL FETAL OXIMETRY PROBE, TRANSCERVICAL FETAL OXIMETRY PROBE, AND/OR TRANSURETHRAL FETAL OXIMETRY PROBE

(71) Applicant: Raydiant Oximetry, Inc., San Ramon, CA (US)

(72) Inventors: NEIL PADHARIA RAY, Sacramento, CA (US); RUSSELL DELONZOR, San Ramon, CA (US)

(21) Appl. No.: 17/912,489

(22) PCT Filed: Mar. 24, 2021
(86) PCT No.: PCT/US2021/024012

§ 371 Date: Sep. 16, 2022

Related U.S. Application Data
(60) Provisional application No. 62/994,058, filed on Mar. 24, 2020.

(51) Int. Cl. A61B 5/1464; A61B 5/00; A61B 5/1459

(52) U.S. Cl. CPC A61B 5/1464; A61B 5/4356; A61B 5/1459

(57) ABSTRACT

Transvaginal and/or transcervical fetal oximetry probes may be configured to take measurements in the endocervical canal of a pregnant mammal that may be used to determine a fetal hemoglobin oxygen saturation level using, for example, oximetry, pulse oximetry, and/or tissue oxygen saturation calculations. Transurethral fetal oximetry probes may be configured to be inserted into a urethra of a pregnant mammal and be positioned proximate to a wall of a bladder of the pregnant mammal proximate to the fetus. Once in position, the Transurethral fetal oximetry probe may take measurements that may be used to determine a fetal hemoglobin oxygen saturation level using, for example, oximetry, pulse oximetry, and/or tissue oxygen saturation calculations.

### RELATED APPLICATION

This patent application is an INTERNATIONAL/PCT application claiming priority to U.S. Provisional Patent Application No. 62/994,058, filed on 24 Mar. 2020 and entitled “SYSTEMS, DEVICES, AND METHODS FOR PERFORMING FETAL OXIMETRY AND/OR FETAL PULSE OXIMETRY USING A TRANSVAGINAL AND/OR TRANSCERVICAL FETAL OXIMETRY PROBE,” which is incorporated in its entirety herein.

### FIELD OF INVENTION

The present invention is in the field of medical devices and, more particularly, in the field of fetal oximetry, fetal pulse oximetry, and fetal tissue oxygenation.

### BACKGROUND

Oximetry is a method for determining the oxygen saturation of hemoglobin in a mammal's blood. Typically, 90% (or higher) of an adult human's hemoglobin is saturated with (i.e., bound to) oxygen while only 30-60% of a fetus's blood is saturated with oxygen. Pulse oximetry is a type of oximetry that uses changes in blood volume through a heartbeat cycle to internally calibrate hemoglobin oxygen saturation measurements of the arterial blood.

Current methods of monitoring fetal health, such as monitoring fetal heart rate, are inefficient at determining levels of fetal distress and, at times, provide false positive results indicating fetal distress that may result in the unnecessary performance of a Cesarean delivery.

### BRIEF DESCRIPTION OF THE FIGURES

The present invention is illustrated by way of example, and not limitation, in the figures of the accompanying drawings in which:

FIG. 1A is a block diagram illustrating an exemplary system for determining a level of oxygen saturation for fetal hemoglobin and/or whether meconium is present in the amniotic fluid of a pregnant mammal, in accordance with some embodiments of the present invention;

FIG. 1B is a block diagram of an exemplary processor-based system that may store data and/or execute instructions for the processes disclosed herein, in accordance with some embodiments of the present invention;

FIG. 1C is a block diagram of an exemplary transabdominal fetal oximetry probe, in accordance with some embodiments of the present invention;

FIG. 2A is a diagram illustrating a cross-section view of a pregnant human woman with a first exemplary transvaginal/transcervical fetal oximetry probe positioned within the pregnant mammal's endocervical canal and proximate to her cervix, in accordance with some embodiments of the present invention;

FIG. 2B is a diagram illustrating the first exemplary transvaginal/transcervical fetal oximetry probe positioned proximate to an approximation of maternal tissue, in accordance with some embodiments of the present invention;

FIG. 2C is a diagram illustrating a cross-section view of a pregnant human woman with the first exemplary transvaginal/transcervical fetal oximetry probe positioned within the pregnant woman's endocervical canal and coincident with the pregnant woman's fetus, in accordance with some embodiments of the present invention;

FIG. 2D is a diagram illustrating the first exemplary transvaginal/transcervical fetal oximetry probe positioned within the pregnant mammal's endocervical canal and proximate to an approximation of a fetus, in accordance with some embodiments of the present invention;

FIG. 2E is a diagram illustrating a cross-section view of a pregnant human woman with a second exemplary transvaginal/transcervical fetal oximetry probe positioned within the pregnant mammal's endocervical canal and proximate to her cervix, in accordance with some embodiments of the present invention;

FIG. 2F is a diagram illustrating the second exemplary transvaginal/transcervical fetal oximetry probe positioned proximate to an approximation of maternal and fetal tissue, in accordance with some embodiments of the present invention;

FIG. 2G is a diagram illustrating a cross-section view of a pregnant human woman with the second exemplary transvaginal/transcervical fetal oximetry probe positioned within the pregnant woman's endocervical canal and coincident with the pregnant woman's fetus, in accordance with some embodiments of the present invention;

FIG. 2H is a diagram illustrating the second exemplary transvaginal/transcervical fetal oximetry probe positioned within the pregnant mammal's endocervical canal and proximate to an approximation of a fetus, in accordance with some embodiments of the present invention;

FIG. 2I is a diagram illustrating a cross-section view of a pregnant human woman with an exemplary transurethral fetal oximetry probe positioned within the pregnant mammal's bladder and proximate to a wall of the bladder closest to her fetus, in accordance with some embodiments of the present invention;

FIG. 2J is a diagram illustrating the exemplary transurethral fetal oximetry probe positioned proximate to an approximation of maternal and fetal tissue, in accordance with some embodiments of the present invention;

FIG. 2K is a diagram illustrating a cross-section view of a pregnant human woman with a first exemplary transurethral fetal oximetry probe/catheter combination positioned within the pregnant mammal's bladder and proximate to a wall of the bladder closest to her fetus, in accordance with some embodiments of the present invention;

FIG. 2L is a diagram illustrating the first exemplary transurethral fetal oximetry probe/catheter combination positioned proximate to an approximation of maternal and fetal tissue, in accordance with some embodiments of the present invention;

FIG. 2M is a diagram illustrating a cross-section view of a pregnant human woman with a second exemplary transurethral fetal oximetry probe/catheter combination positioned within the pregnant mammal's bladder and proximate to a wall of the bladder closest to her fetus, in accordance with some embodiments of the present invention;

FIG. 2N is a diagram illustrating the second exemplary transurethral fetal oximetry probe/catheter combination positioned proximate to an approximation of maternal and fetal tissue, in accordance with some embodiments of the present invention;

FIG. 2O is a diagram of a cross section of the second exemplary transurethral fetal oximetry probe/catheter combination, in accordance with some embodiments of the present invention;

FIG. 3 is a flowchart illustrating an exemplary process for performing fetal oximetry and/or fetal pulse oximetry and/or determining fetal tissue oxygen saturation using a transvaginal/transcervical fetal oximetry probe, in accordance with some embodiments of the present invention;

FIG. 4 is a flowchart illustrating an exemplary process for performing fetal oximetry and/or fetal pulse oximetry and/or determining fetal tissue oxygen saturation using both a transabdominal fetal oximetry probe and a transvaginal and/or transcervical fetal oximetry probe, in accordance with some embodiments of the present invention;

FIG. 5 is a flowchart illustrating an exemplary process for performing fetal oximetry and/or fetal pulse oximetry and/or determining fetal tissue oxygen saturation using both a transabdominal fetal oximetry probe and a transvaginal/transcervical fetal oximetry probe, in accordance with some embodiments of the present invention;

FIG. 6 is a flowchart illustrating a process for verifying a determination of fetal hemoglobin and/or tissue oxygen saturation made by a transabdominal fetal oximetry probe using a transvaginal/transcervical fetal oximetry probe, in accordance with some embodiments of the present invention; and

FIG. 7 is a flowchart illustrating a process for determining an overall fetal hemoglobin using a detected electronic signal from a transabdominal fetal oximetry probe and a detected electronic signal from a transvaginal/transcervical fetal oximetry probe, in accordance with some embodiments of the present invention.

Throughout the drawings, the same reference numerals and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### SUMMARY

Systems, devices, and methods for performing fetal oximetry and/or fetal pulse oximetry using a transvaginal fetal oximetry probe, transcervical fetal oximetry probe, and/or transurethral fetal oximetry probe are described herein. Exemplary transvaginal fetal oximetry probes, transcervical fetal oximetry probes, and transurethral fetal oximetry probes may include a light source, one or more detectors, and a housing. For transvaginal fetal oximetry probes and transcervical fetal oximetry probes, the light source may be configured to project light of a plurality of wavelengths into the endocervical canal of a pregnant mammal to be incident on a fetus within the pregnant mammal's abdomen. When the transvaginal fetal oximetry probe is positioned on the outside of the cervix, the light from the light source may be incident upon the cervical tissue and other maternal tissue and/or amniotic fluid positioned between the light source and the fetus. When the transcervical fetal oximetry probe is positioned directly on the fetus when, for example, the cervix is sufficiently dilated to allow for passage of the transcervical fetal oximetry probe through the dilated cervix and positioning of the transcervical fetal oximetry probe directly on the fetus, often times the fetus' head, the light from the light source may be incident directly upon the fetus. For transurethral fetal oximetry probes, the light source may be configured to project light onto the maternal tissue (e.g., bladder and uterine walls) positioned between the transurethral fetal oximetry probes and the fetus.

Detectors included in transvaginal fetal oximetry probes, transcervical fetal oximetry probes, and transurethral fetal oximetry probes may be configured to detect light reflected from the fetus and, in the case of the transvaginal and transurethral fetal oximetry probes, pregnant mammal's tissue and convert the detected light into one or more detected electronic signals that may be communicated to an external processor configured to determine a level of fetal hemoglobin oxygen saturation and/or fetal tissue oxygen saturation with the detected electronic signal.

The transvaginal fetal oximetry probes, transcervical fetal oximetry probes, and transurethral fetal oximetry probes disclosed herein also include a housing configured to house the light source and the one or more detectors. The housings of the transvaginal fetal oximetry probes, transcervical fetal oximetry probes, and/or transurethral fetal oximetry probes may be configured, sized, and shaped so that they are easily inserted into an endocervical canal or urethra of the pregnant mammal. In some cases, the housing may be flexible so that it may bend with the curves and shape of the pregnant mammal's anatomy. In some embodiments, a shape and/or form factor for transvaginal fetal oximetry probes and transcervical fetal oximetry probes disclosed herein may be similar and/or the same. In some cases, a transvaginal fetal oximetry probe may be used transcervically when, for example, the cervix has dilated enough to allow for the passage of the transvaginal fetal oximetry probe through the dilated cervix so that it may be positioned directly on the fetus.

In some instances, the housings of the transvaginal fetal oximetry probes, transcervical fetal oximetry probes, and/or transurethral fetal oximetry probes may include a cord that extends from the housing and may be configured to electrically couple the transvaginal fetal oximetry probe to a power source (thereby providing electrical power to the light source and one or more detectors) and/or communicate the detected electronic signal from the detector to the external processor. In some embodiments, the cord may be configured to facilitate extraction of the transvaginal fetal oximetry probe and/or transcervical fetal oximetry probe from the pregnant mammal's endocervical canal and/or extract the transurethral fetal oximetry probe from the pregnant mammal's urethra/bladder. Additionally, or alternatively, the transvaginal fetal oximetry probes, transcervical fetal oximetry probes, and/or transurethral fetal oximetry probes disclosed herein may include a power source within the housing such as a battery that in some cases may be rechargeable. In some instances, the transvaginal fetal oximetry probes, transcervical fetal oximetry probes, and/or transurethral fetal oximetry probes may be configured to wirelessly communicate with the external processor via, for example, a transceiver that may be, for example, Wi-Fi and/or Bluetooth enabled.

In some embodiments, the transvaginal fetal oximetry probes, transcervical fetal oximetry probes, and/or transurethral fetal oximetry probes disclosed herein may include a processing device (e.g., a CPU, an application-specific integrated circuit (ASIC), and/or a processor) configured to pre-process the detected electronic signal. The preprocessing may include filtering the signal to reduce noise and/or filter out artifacts in the signal caused by, for example, maternal movement and/or equipment noise that may interfere with the clarity of the detected electronic signals.

The methods disclosed herein may include receiving a first detected electronic signal from a transabdominal fetal oximetry probe, determining a first fetal hemoglobin oxygen saturation level using the first detected electronic signal, receiving a second detected electronic signal from a transvaginal fetal oximetry probe, and then determining a second fetal hemoglobin oxygen saturation level using the second detected electronic signal. The first fetal hemoglobin oxygen saturation level may then be compared to the second fetal hemoglobin oxygen saturation level and it may be determined whether the first fetal hemoglobin oxygen saturation level and the second fetal hemoglobin oxygen saturation level are within a specified range of values and, if so, an indication of the first and second fetal hemoglobin oxygen saturation level may be provided to a user.

On some occasions, the first detected electronic signal may be timestamped and the determining the first fetal hemoglobin oxygen saturation level may include receiving a timestamped maternal heart beat signal, synchronizing the maternal heart beat signal and the first detected electronic signal using a timestamp of the maternal heart beat signal and a timestamp first detected electronic signal, isolating a fetal signal from the detected electronic signal by subtracting portions of the first detected electronic signal that correspond to the maternal heart beat signal, and calculating the first fetal hemoglobin oxygen saturation level using the fetal signal. Additionally, or alternatively, the second detected electronic signal may be timestamped and determining the second fetal hemoglobin oxygen saturation level may include receiving a timestamped maternal heart beat signal, synchronizing the maternal heart beat signal and the second detected electronic signal using a timestamp of the maternal heart beat signal and a timestamp second detected electronic signal, isolating a fetal signal from the second detected electronic signal by subtracting portions of the second detected electronic signal that correspond to the maternal heart beat signal, and calculating the second fetal hemoglobin oxygen saturation level using the fetal signal.

Additionally, or alternatively, the first detected electronic signal may be timestamped and the determining of the first fetal hemoglobin oxygen saturation level may include receiving a timestamped fetal heart beat signal, synchronizing the fetal heart beat signal and the first detected electronic signal using a timestamp of the fetal heart beat signal and a timestamp first detected electronic signal, isolating a fetal signal from the first detected electronic signal by amplifying portions of the first detected electronic signal that correspond to the fetal heart beat signal, and calculating the first fetal hemoglobin oxygen saturation level using the fetal signal.

Additionally, or alternatively, the second detected electronic signal may be timestamped and the determining of the second fetal hemoglobin oxygen saturation level may include receiving a timestamped fetal heart beat signal, synchronizing the fetal heart beat signal and the second detected electronic signal using a timestamp of the fetal heart beat signal and a timestamp second detected electronic signal, isolating a fetal signal from the second detected electronic signal by amplifying portions of the second detected electronic signal that correspond to the fetal heart beat signal, and calculating the second fetal hemoglobin oxygen saturation level using the fetal signal.

In some embodiments, a characteristic of the pregnant mammal may be received and the received characteristic may be used to determine the first and/or second fetal hemoglobin oxygen saturation level. Exemplary characteristics include, but are not limited to, maternal hemoglobin oxygen saturation level, maternal heart rate, thickness of maternal tissue the light passes through, type of maternal tissue the light passes through, maternal blood pressure, and maternal respiratory rate.

In some embodiments, a first detected electronic signal may be received from a transabdominal fetal oximetry probe and a first fetal hemoglobin oxygen saturation level may be determined using the first detected electronic signal. A second detected electronic signal may be received from a transvaginal fetal oximetry probe and a second fetal hemoglobin oxygen saturation level may be determined using the second detected electronic signal. Then, an overall fetal hemoglobin oxygen saturation level may be determined using the first fetal hemoglobin oxygen saturation level and the second fetal hemoglobin oxygen saturation level and an indication of the overall fetal hemoglobin oxygen saturation level may be provided to a user.

In some embodiments, the first detected electronic signal may be timestamped and the determining of the first fetal hemoglobin oxygen saturation level may include receiving a timestamped maternal heart beat signal, synchronizing the maternal heart beat signal and the first detected electronic signal using a timestamp of the maternal heart beat signal and a timestamp first detected electronic signal, isolating a fetal signal from the detected electronic signal by subtracting portions of the first detected electronic signal that correspond to the maternal heart beat signal, and calculating the first fetal hemoglobin oxygen saturation level using the fetal signal. Additionally, or alternatively, determining of the first fetal hemoglobin oxygen saturation level may include receiving a timestamped fetal heart beat signal, synchronizing the fetal heart beat signal and the first detected electronic signal using a timestamp of the fetal heart beat signal and a timestamp first detected electronic signal, isolating a fetal signal from the first detected electronic signal by amplifying portions of the first detected electronic signal that correspond to the fetal heart beat signal, and calculating the first fetal hemoglobin oxygen saturation level using the fetal signal.

Additionally, or alternatively, the second detected electronic signal may be timestamped and the determining of the second fetal hemoglobin oxygen saturation level may include receiving a timestamped maternal heart beat signal, synchronizing the maternal heart beat signal and the second detected electronic signal using a timestamp of the maternal heart beat signal and a timestamp second detected electronic signal, isolating a fetal signal from the second detected electronic signal by subtracting portions of the second detected electronic signal that correspond to the maternal heart beat signal, and calculating the second fetal hemoglobin oxygen saturation level using the fetal signal. Additionally, or alternatively, determining of the second fetal hemoglobin oxygen saturation level may include receiving a timestamped fetal heart beat signal, synchronizing the fetal heart beat signal and the second detected electronic signal using a timestamp of the fetal heart beat signal and a timestamp second detected electronic signal, isolating a fetal signal from the second detected electronic signal by amplifying portions of the second detected electronic signal that correspond to the fetal heart beat signal, and calculating the second fetal hemoglobin oxygen saturation level using the fetal signal.

Additionally, or alternatively, a characteristic of the pregnant mammal may be received and the first and/or second fetal hemoglobin oxygen saturation level may be determined using the characteristic of the pregnant mammal. The characteristic of the pregnant mammal may be, for example, maternal hemoglobin oxygen saturation level, maternal heart rate, thickness of maternal tissue the light passes through, type of maternal tissue the light passes through, maternal blood pressure, and/or maternal respiratory rate.

In another embodiment, a first detected electronic signal may be received from a transabdominal fetal oximetry probe and a first fetal hemoglobin oxygen saturation level may be determined using the first detected electronic signal. A second detected electronic signal may be received from a transurethral fetal oximetry probe and a second fetal hemoglobin oxygen saturation level may be determined using the second detected electronic signal. The first and second fetal hemoglobin oxygen saturation levels may be compared and it may be determined whether the first fetal hemoglobin oxygen saturation level and the second fetal hemoglobin oxygen saturation level are within a specified range of values (e.g., within a standard of deviation from one another) and an indication of the comparison and/or a value for the first and/or second fetal hemoglobin oxygen saturation level may be provided to a user.

In another embodiment, a first detected electronic signal may be received from a transabdominal fetal oximetry probe and a first fetal hemoglobin oxygen saturation level may be determined using the first detected electronic signal. A second detected electronic signal may be received from a transurethral fetal oximetry probe and a second fetal hemoglobin oxygen saturation level may be determined using the second detected electronic signal. Then, an overall fetal hemoglobin oxygen saturation level may be determined using the first fetal hemoglobin oxygen saturation level and the second fetal hemoglobin oxygen saturation level and an indication of the overall fetal hemoglobin oxygen saturation level may be provided to a user.

### Description

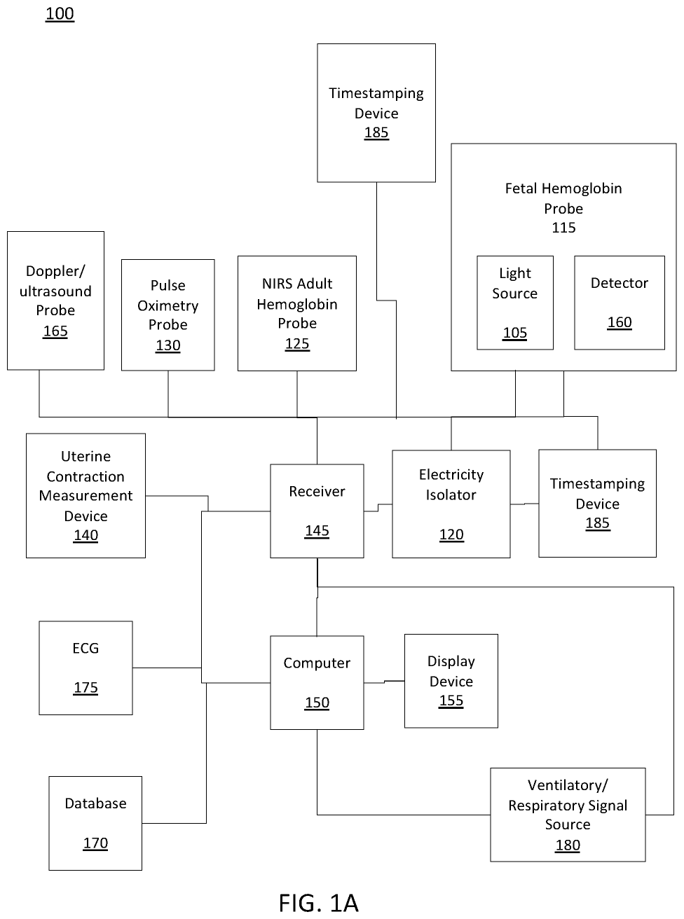

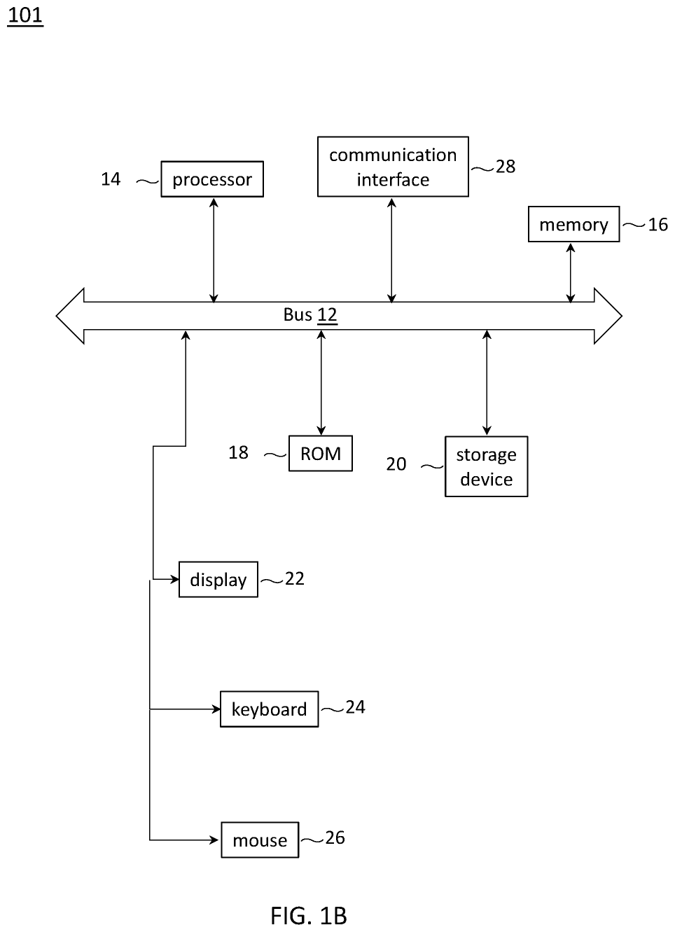

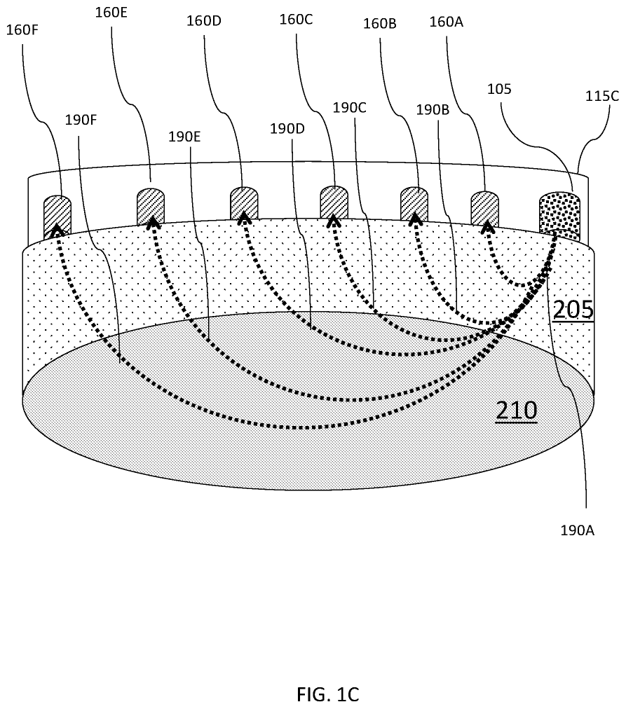

FIG. 1 provides an exemplary system 100 for detecting and/or determining fetal hemoglobin oxygen saturation levels and/or fetal depth. The components of system 100 may be coupled together via wired and/or wireless communication links. In some instances, wireless communication of one or more components of system 100 may be enabled using short-range wireless communication protocols designed to communicate over relatively short distances (e.g., BLUETOOTH™, near field communication (NFC), radio-frequency identification (RFID), and Wi-Fi) with, for example, a computer or personal electronic device (e.g., tablet computer or smart phone) as described below.

System 100 includes a fetal oximetry probe 115 that includes at least one light source 105 and at least one a detector 160. On some occasions, fetal oximetry probe 115 may include a power source such as a battery and/or port by which to couple fetal oximetry probe 115 to a power source such as an outlet. Light source 105 may include a single, or multiple light sources and detector 160 may include a single, or multiple detectors. Light source 105 may transmit light of one or more wavelengths, including near infra-red (NIR), into the pregnant mammal's abdomen. Typically, the light emitted by light source 105 is focused or emitted as a narrow beam to reduce spreading of the light upon entry into the pregnant mammal's abdomen. Light source 105 may be, for example, a LED, and/or a LASER that may be coupled to a fiber optic cable. On some occasions, the light sources may be one or more fiber optic cables optically coupled to a laser and arranged in an array. In some instances, light source 105 may be tunable or otherwise user configurable while, in other instances, light source 105 may be configured to emit light within a pre-defined range of wavelengths. Additionally, or alternatively, one or more filters (not shown) and/or polarizers may filter/polarize the light emitted by light sources 105 to be of one or more preferred wavelengths. In some cases, these filters/polarizers may also be tunable or user configurable.

An exemplary light source 105 may have a relatively small form factor and may operate with high efficiency, which may serve to, for example, conserve space and/or limit heat emitted by the light source 105. In one embodiment, light source 105 is configured to emit light in the range of 770-850 nm. In some embodiments, light source 105 (or multiple light sources 105) may emit light of at least two different frequencies (e.g., 600 nm and 900 nm; 735 and 890 nm; 670 nm and 700 nm; 735 nm and 850 nm; or 850 nm and 890 nm). Exemplary flux ratios for light sources include but are not limited to a luminous flux/radiant flux of 175-260 mW, a total radiant flux of 300-550 mW and a power rating of 0.6 W-3.5 W. A power for a light source 105 may be approximately 200 mWcm⁻².

Detector 160 may be a detector configured to detect light emanating from the pregnant mammal and/or the fetus via, for example, transmission and/or back scattering and convert this light signal into an electronic signal, which may be referred to herein as a composite signal and/or a detected electronic signal. The detected electronic signal may be communicated to a computer or processor such as computer 150 and/or a receiver such as receiver 145 via, for example, an on-board transceiver and/or a wired communication link.

Exemplary detectors 160 include, but are not limited to, photodetectors, cameras, traditional photomultiplier tubes (PMTs), silicon PMTs, avalanche photodiodes, and silicon photodiodes. In some embodiments, the detectors will have a relatively low cost (e.g., $50 or below), a low voltage requirement (e.g., less than 100 volts), and non-glass (e.g., plastic) form factor. In other embodiments, (e.g., contactless pulse oximetry) a sensitive camera may be deployed to receive light emitted by the pregnant mammal's abdomen. For example, detector 160 may be a sensitive camera adapted to capture small changes in fetal skin tone caused by changes in cardiovascular pressure associated with fetal myocardial contractions. In these embodiments, detector 160 and/or fetal oximetry probe 115 may be in contact with the pregnant mammal's abdomen, or not, as this embodiment may be used to perform so-called contactless pulse oximetry. In these embodiments, light source 105 may be adapted to provide light (e.g., in the visible spectrum, near-infrared, etc.) directed toward the pregnant mammal's abdomen so that the detector 160 is able to receive/detect light emitted by the pregnant mammal's abdomen and fetus.

An exemplary quantity of photons produced by light source 105 is 0.5-2 billion per cycle or for each emission of light. In some cases, the emitted light may be modulated.

In some cases, fetal oximetry probe 115 may be a transabdominal fetal oximetry probe configured to be affixed to the epidermis of the pregnant mammal's abdomen. An exemplary transabdominal fetal oximetry probe is provided in FIG. 1C and discussed below.

System 100 includes a number of optional independent sensors/probes designed to monitor various aspects of maternal and/or fetal health. These probes/sensors are a NIRS adult hemoglobin probe 125, a pulse oximetry probe 130, a Doppler and/or ultrasound probe 135, a uterine contraction measurement device 140, an electrocardiography (ECG) machine 175, and a ventilatory/respiratory signal source 180.

ECG 175 may be used to determine the pregnant mammal's and/or fetus's heart rate. In some embodiments, ECG 175 may be a fetal ECG that may be used to determine the fetus's heart rate. In some instances, ECG 175 may be used internally via, for example, placement in the endocervical canal. At times, placement of ECG 175 in the endocervical canal may be facilitated by inclusion in a transvaginal and/or transcervical probe as disclosed herein.

Doppler and/or ultrasound probe 135 may be configured to be placed on the abdomen of the pregnant mammal and may provide information regarding, for example, fetal depth, fetal position, orientation, and/or heart rate. Pulse oximetry probe 130 may be a conventional pulse oximetry probe placed on, for example, the pregnant mammal's earlobe and/or finger to measure the pregnant mammal's hemoglobin oxygen saturation level. NIRS adult hemoglobin probe 125 may be placed on, for example, the pregnant mammal's 2nd finger and may be configured to, for example, use near infrared spectroscopy to calculate the ratio of adult oxyhemoglobin to adult de-oxyhemoglobin. NIRS adult hemoglobin probe 125 may also be used to determine the pregnant mammal's heart rate.

Optionally, system 100 may include a uterine contraction measurement device 140 configured to measure the strength and/or timing of the pregnant mammal's uterine contractions. In some embodiments, uterine contractions may be measured by uterine contraction measurement device 140 as a function of pressure (e.g., measured in e.g., mmHg) over time. In some instances, uterine contraction measurement device 140 is and/or includes a tocotransducer, which is an instrument that includes a pressure-sensing area that detects changes in the abdominal contour to measure uterine activity and, in this way, monitors frequency and duration of contractions.

In another embodiment, uterine contraction measurement device 140 may be configured to pass an electrical current through the pregnant mammal and measure changes in the electrical current as the uterus contracts. Additionally, or alternatively, uterine contractions may also be measured via near infrared spectroscopy using, for example, light received/detected by detector 160 because uterine contractions, which are muscle contractions, are oscillations of the uterine muscle between a contracted state and a relaxed state. Oxygen consumption of the uterine muscle during both of these stages is different and these differences may be detectable using NIRS.

Measurements and/or signals from NIRS adult hemoglobin probe 125, pulse oximetry probe 130, Doppler and/or ultrasound probe 135, and/or uterine contraction measurement device 140 may be communicated directly to computer 150 and/or to receiver 145 for communication to computer 150 and display on display device 155 and, in some instances, may be considered secondary signals. In some embodiments, measurements provided by NIRS adult hemoglobin probe 125, pulse oximetry probe 130, a Doppler and/or ultrasound probe 135, uterine contraction measurement device 140, ECG 175, and/or ventilatory/respiratory signal source 180 may be used in conjunction with fetal oximetry probe 115 to isolate a fetal pulse signal and/or fetal heart rate from a maternal pulse signal and/or maternal heart rate. Receiver 145 may be configured to receive signals and/or data from one or more components of system 100 including, but not limited to, fetal oximetry probe 115, NIRS adult hemoglobin probe 125, pulse oximetry probe 130, Doppler and/or ultrasound probe 135, uterine contraction measurement device 140, ECG 175, and/or ventilatory/respiratory signal source 180. Communication between receiver 145 and/or computer 150 and other components of system 100 may be made using wired or wireless communication.

In some instances, one or more of NIRS adult hemoglobin probe 125, pulse oximetry probe 130, a Doppler and/or ultrasound probe 135, uterine contraction measurement device 140, ECG 175, and/or ventilatory/respiratory signal source 180 may include a dedicated display that provides the measurements to, for example, a user or medical treatment provider. It is important to note that not all of these probes are used in every instance. For example, when the pregnant mammal is using fetal oximetry probe 115 in a setting outside of a hospital or treatment facility (e.g., at home or work) then, some of the probes (e.g., NIRS adult hemoglobin probe 125, pulse oximetry probe 130, a Doppler and/or ultrasound probe 135, uterine contraction measurement device 140, ECG 175, and/or ventilatory/respiratory signal source 180) of system 100 may not be used.

In some instances, receiver 145 may be configured to process or pre-process received signals so as to, for example, make the signals compatible with computer 150 (e.g., convert an optical signal to an electrical signal), improve signal to noise ratio (SNR), amplify a received signal, etc. In some instances, receiver 145 may be resident within and/or may be a component of computer 150. In some embodiments, computer 150 may amplify or otherwise condition the received detected signal so as to, for example, improve the signal-to-noise ratio.

Receiver 145 may communicate received, pre-processed, and/or processed signals to computer 150. Computer 150 may act to process the received signals, as discussed in greater detail below, and facilitate provision of the results to a display device 155. Exemplary computers 150 include desktop and laptop computers, servers, tablet computers, personal electronic devices, mobile devices (e.g., smart phones), and the like. Exemplary display devices 155 are computer monitors, tablet computer devices, and displays provided by one or more of the components of system 100. In some instances, display device 155 may be resident in receiver 145 and/or computer 150. Computer 150 may be communicatively coupled to a database 170, which may be configured to store information regarding physiological characteristic and/or combinations of physiological characteristic of pregnant mammals and/or their fetuses, impacts of physiological characteristic on light behavior, information regarding the calculation of hemoglobin oxygen saturation levels, calibration factors, and so on.

In some embodiments, a pregnant mammal may be electrically insulated from one or more components of system 100 by, for example, an electricity isolator 120. Exemplary electricity insulators 120 include circuit breakers, ground fault switches, and fuses.

In some embodiments, system 100 may include a ventilatory/respiratory signal source 180 that may be configured to monitor the pregnant mammal's respiratory rate and provide a respiratory signal indicating the pregnant mammal's respiratory rate to, for example, computer 150. Additionally, or alternatively, ventilatory/respiratory signal source 180 may be a source of a ventilatory signal obtained via, for example, cooperation with a ventilation machine. Exemplary ventilatory/respiratory signal sources 180 include, but are not limited to, a carbon dioxide measurement device, a stethoscope and/or electronic acoustic stethoscope, a device that measures chest excursion for the pregnant mammal, and a pulse oximeter. A signal from a pulse oximeter may be analyzed to determine variations in the PPG signal that may correspond to respiration for the pregnant mammal. Additionally, or alternatively, ventilatory/respiratory signal source 180 may provide a respiratory signal that corresponds to a frequency with which gas (e.g., air, anesthetic, etc.) is provided to the pregnant mammal during, for example, a surgical procedure. This respiratory signal may be used to, for example, determine a frequency of respiration for the pregnant mammal.

In some embodiments, system 100 may include a timestamping device 185. Timestamping device 185 may be configured to timestamp a signal generated and/or provided by, for example, fetal oximetry probe 115, Doppler/ultrasound probe 135, pulse oximetry probe 130, NIRS adult hemoglobin probe, uterine contraction measurement device 140, ECG 175, and/or ventilatory/respiratory signal source 180 with a timestamp that represents, for example, an event (e.g., time, or t, =0, 10, 20, etc.) and/or chronological time (e.g., date and time). Timestamping device 185 may timestamp a signal via, for example, introducing a ground signal into system 100 that may simultaneously, or nearly simultaneously, interrupt or otherwise introduce a stamp or other indicator into a signal generated by one or more of, for example, fetal oximetry probe 115, Doppler/ultrasound probe 135, pulse oximetry probe 130, NIRS adult hemoglobin probe, uterine contraction measurement device 140, ECG 175, and/or ventilatory/respiratory signal source 180. Additionally, or alternatively, timestamping device 185 may timestamp a signal via, for example, introducing an optical signal into system 100 that may simultaneously, or nearly simultaneously, interrupt or otherwise introduce a stamp or other indicator into a signal generated by one or more of, for example, fetal oximetry probe 115, pulse oximetry probe 130, NIRS adult hemoglobin probe, uterine contraction measurement device 140. Additionally, or alternatively, timestamping device 185 may timestamp a signal via, for example, introducing an acoustic signal into system 100 that may simultaneously, or nearly simultaneously, interrupt or otherwise introduce a stamp or other indicator into a signal generated by one or more of, for example, fetal oximetry probe 115, Doppler/ultrasound probe 135, and/or ventilatory/respiratory signal source 180.

A timestamp generated by timestamping device 185 may serve as a simultaneous, or nearly simultaneous starting point, or benchmark, for the processing, measuring, synchronizing, correlating, and/or analyzing of a signal from, for example, fetal oximetry probe 115, Doppler/ultrasound probe 135, pulse oximetry probe 130, NIRS adult hemoglobin probe 125, uterine contraction measurement device 140, ECG 175, and/or ventilatory/respiratory signal source 180. In some instances, a timestamp may be used to correlate and/or synchronize two or more signals generated by, for example, fetal oximetry probe 115, Doppler/ultrasound probe 135, pulse oximetry probe 130, NIRS adult hemoglobin probe, uterine contraction measurement device 140, ECG 175, and/or ventilatory/respiratory signal source 180 so that, for example, they align in the time domain.

FIG. 1B provides an example of a processor-based system 101 that may store and/or execute instructions for one or more of the processes described herein. Processor-based system 101 may be representative of, for example, computing device 1450 and/or the components of housing 125 and/or 805. Note, not all of the various processor-based systems which may be employed in accordance with embodiments of the present invention have all of the features of system 101. For example, certain processor-based systems may not include a display inasmuch as the display function may be provided by a client computer communicatively coupled to the processor-based system or a display function may be unnecessary. Such details are not critical to the present invention.

System 101 includes a bus 12 or other communication mechanism for communicating information, and a processor 14 coupled with the bus 12 for processing information. System 101 also includes a main memory 16, such as a random-access memory (RAM) or other dynamic storage device, coupled to the bus 12 for storing information and instructions to be executed by processor 14. Main memory 16 also may be used for storing temporary variables or other intermediate information during execution of instructions to be executed by processor 14. System 101 further includes a read only memory (ROM) 18 or other static storage device coupled to the bus 12 for storing static information and instructions for the processor 14. A storage device 20, which may be one or more of a hard disk, flash memory-based storage medium, a magnetic storage medium, an optical storage medium (e.g., a Blu-ray disk, a digital versatile disk (DVD)-ROM), or any other storage medium from which processor 14 can read, is provided and coupled to the bus 12 for storing information and instructions (e.g., operating systems, applications programs and the like).

System 101 may be coupled via the bus 12 to a display 22, such as a flat panel display, for displaying information to a user. An input device 24, such as a keyboard including alphanumeric and other keys, may be coupled to the bus 12 for communicating information and command selections to the processor 14. Another type of user input device is cursor control device 26, such as a mouse, a trackball, or cursor direction keys for communicating direction information and command selections to processor 14 and for controlling cursor movement on the display 22. Other user interface devices, such as microphones, speakers, etc. are not shown in detail but may be involved with the receipt of user input and/or presentation of output.

The processes referred to herein may be implemented by processor 14 executing appropriate sequences of processor-readable instructions stored in main memory 16. Such instructions may be read into main memory 16 from another processor-readable medium, such as storage device 20, and execution of the sequences of instructions contained in the main memory 16 causes the processor 14 to perform the associated actions. In alternative embodiments, hard-wired circuitry or firmware-controlled processing units (e.g., field programmable gate arrays) may be used in place of or in combination with processor 14 and its associated computer software instructions to implement the invention. The processor-readable instructions may be rendered in any computer language.

System 101 may also include a communication interface 28 coupled to the bus 12. Communication interface 28 may provide a two-way data communication channel with a computer network, which provides connectivity to the plasma processing systems discussed above. For example, communication interface 28 may be a local area network (LAN) card to provide a data communication connection to a compatible LAN, which itself is communicatively coupled to other computer systems. The precise details of such communication paths are not critical to the present invention. What is important is that system 101 can send and receive messages and data through the communication interface 28 and in that way communicate with other controllers, etc.

FIG. 1C illustrates an exemplary fetal probe 115C positioned on a pregnant mammal's abdomen. The maternal tissue of the pregnant mammal's abdomen is represented as an abstraction of maternal tissue 205 and a fetus within the pregnant mammal's abdomen is represented as an abstraction of a fetus 210.

Fetal probe 115C has one light source 105 and six detectors 160A, 160B, 160C, 160D, 160E, and 160F, each of which have a different position relative to source 105 with first detector 160 A being the closest to source 105 and sixth detector 160F being the furthest away from source 105. A position of a detector 160A-160F relative to source 105 may be referred to herein as a source/detector distance. In some examples, detectors 160A-160F may be arranged linearly and may be positioned 1 cm apart from one another so that first detector 160A is positioned 1 cm away from source 105, second detector 160B is positioned 1 cm away from first detector 160A, third detector 160C is positioned 1 cm away from second detector 160B, fourth detector 160D is positioned 1 cm away from third detector 160C, fifth detector 160E is positioned 1 cm away from fourth detector 160D, and sixth detector 160F is positioned 1 cm away from fifth detector 160E.

Source 105 may project an optical signal 190 into the pregnant mammal's abdomen 205 and a resultant optical signal may be detected by one or more of detector(s) 160A-160F. It is expected that the detectors positioned closer to source 105 will detect a portion of the optical signal that has been incident on the pregnant mammal's abdomen 205 but not fetus 210 and, in some embodiments, first detector 160A and/or second detector 160B may be positioned via, for example, setting of a source/detector distance, so that a majority, if not all, of an optical signal 190A and 190B detected by first and second detectors 160A and 160B, respectively, has only been incident of the pregnant mammal's abdomen 205 (i.e., is not incident on the fetus). Third-sixth detectors 160C-160F may detect portions of the optical signal 190C, 190D, 190E, and 190F that are incident on the pregnant mammal 205 and fetus 210 as shown in FIG. 1C. In some cases, third detector 160C may be positioned 3-5 cm away from the light source and sixth detector 160F may be positioned 6-10 cm away from the light source. Additionally or alternatively, third-sixth detectors 160C-160F may be positioned within 4-10 cm of the light source.

As the source/detector distance increases a proportion of the optical signal that corresponds to light that was incident on fetus 210 increases. Thus, optical signal 190F may include a higher proportion of light that was incident on the fetus than, for example, optical signal 190E or 190D.

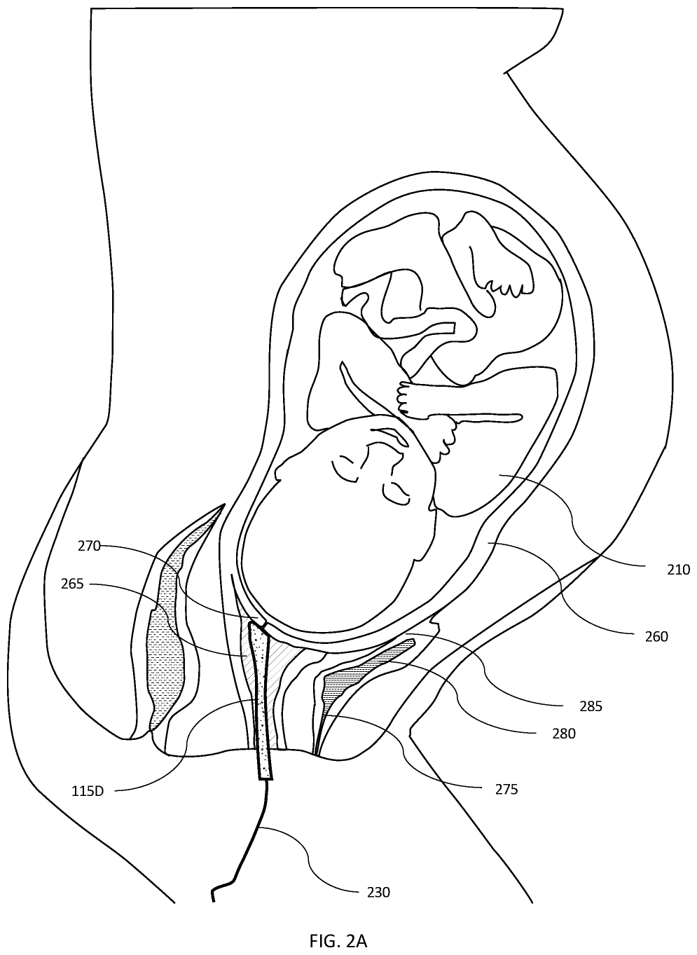

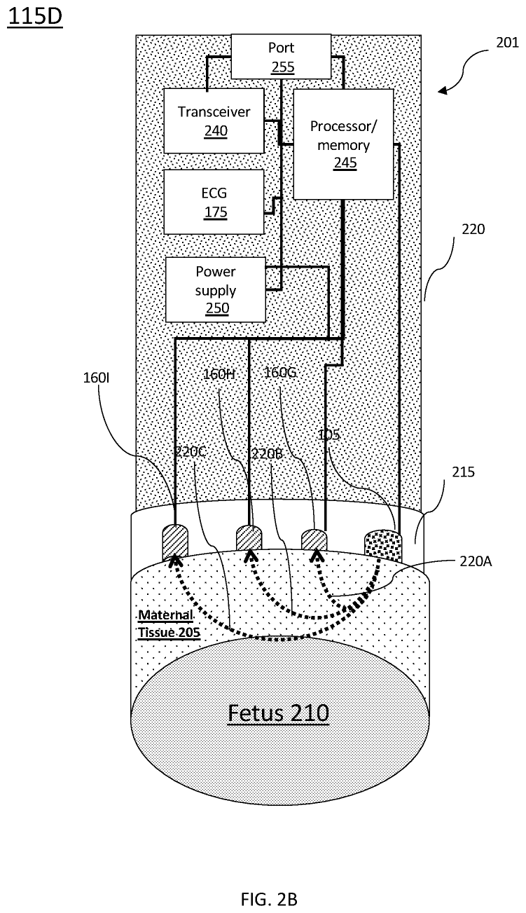

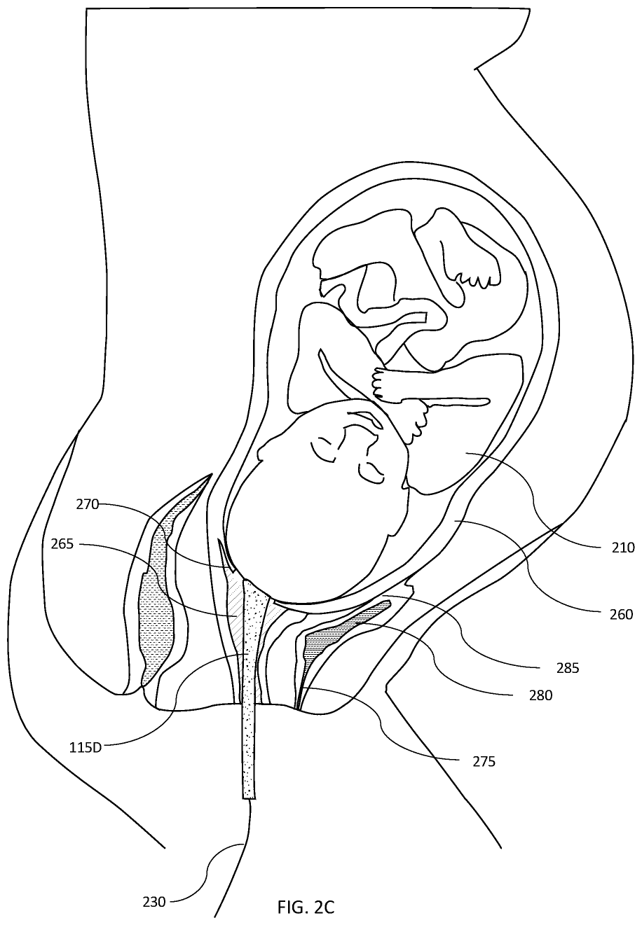

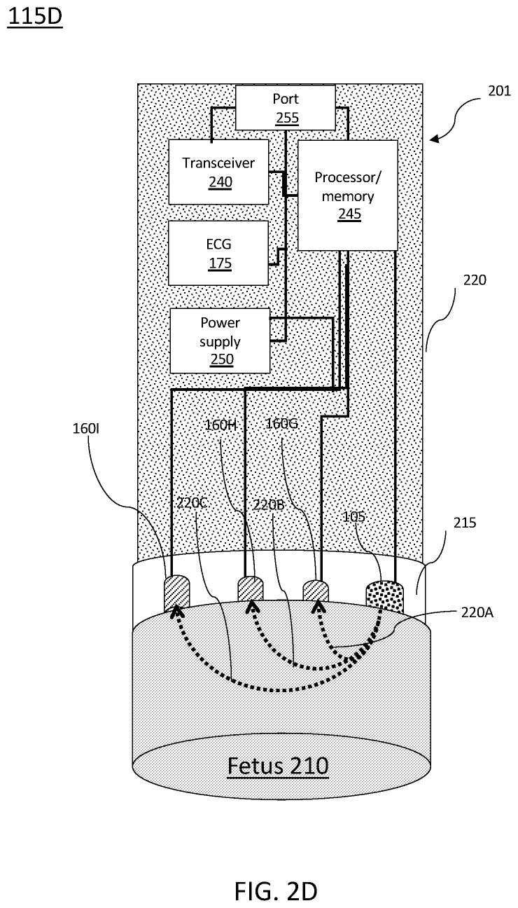

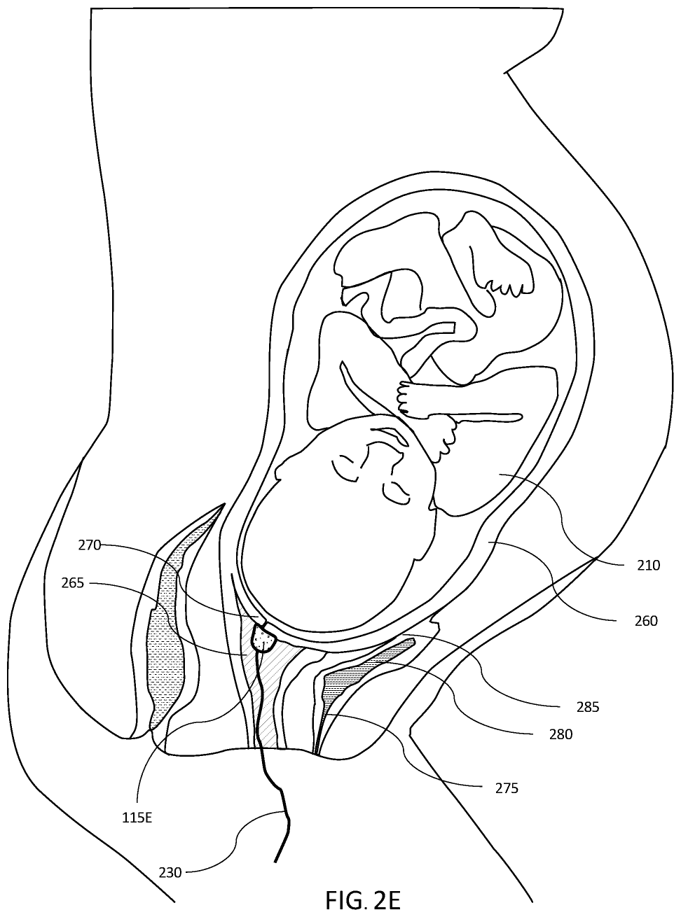

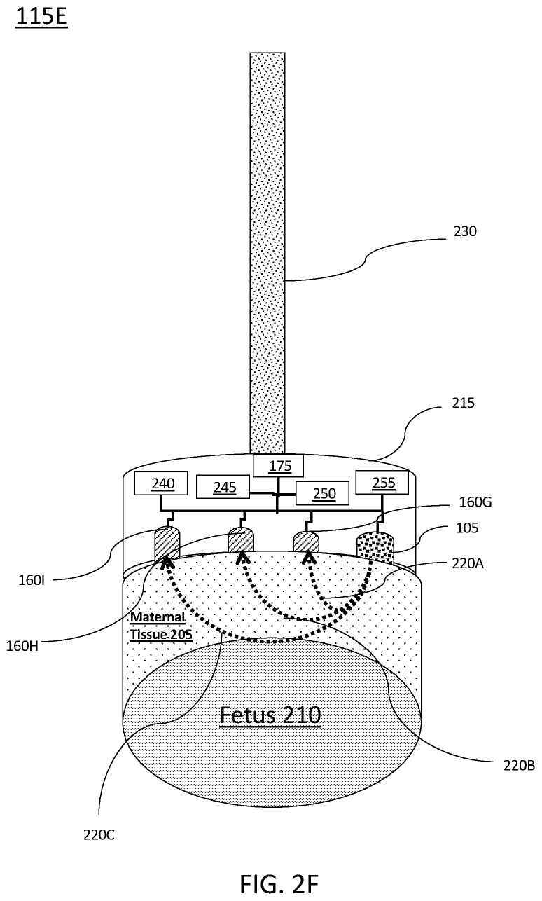

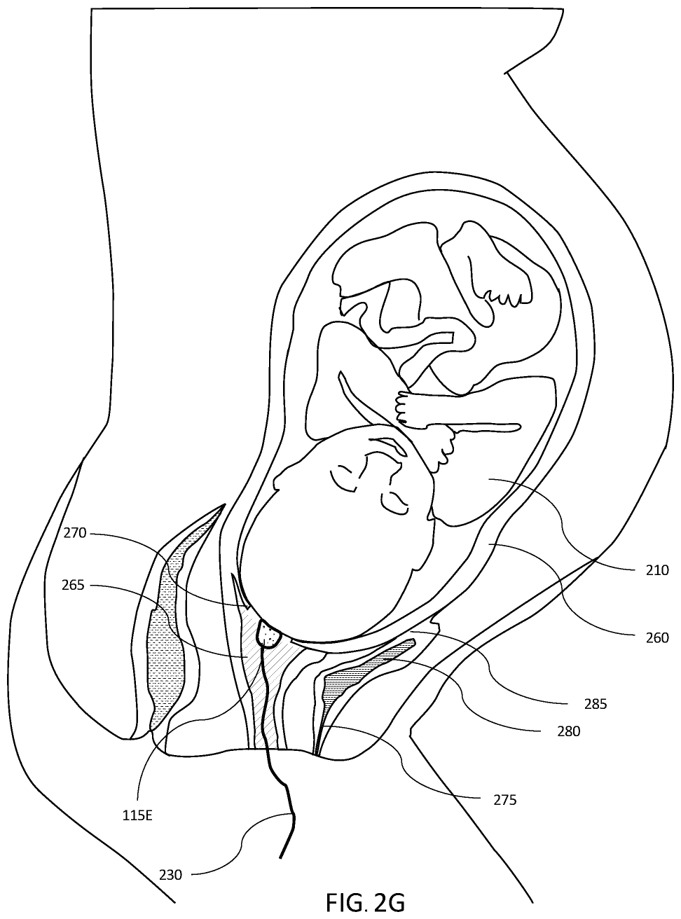

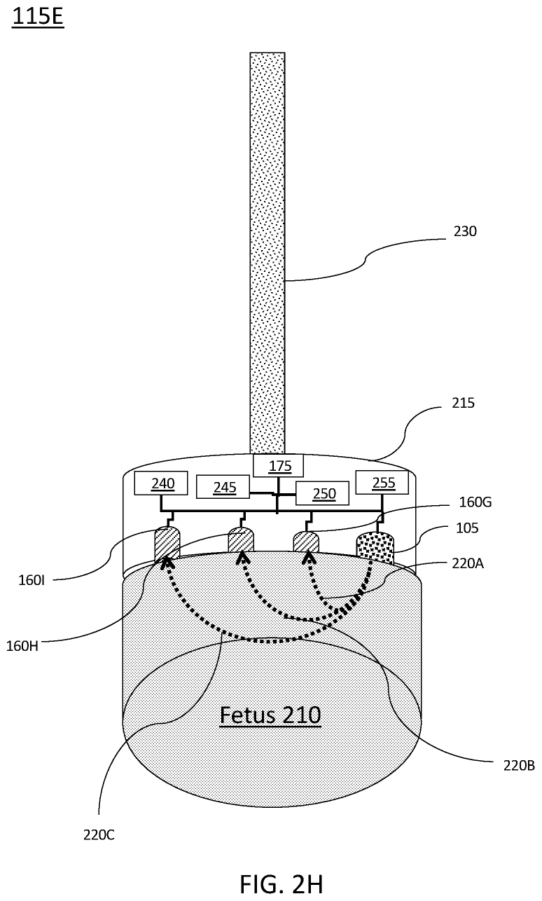

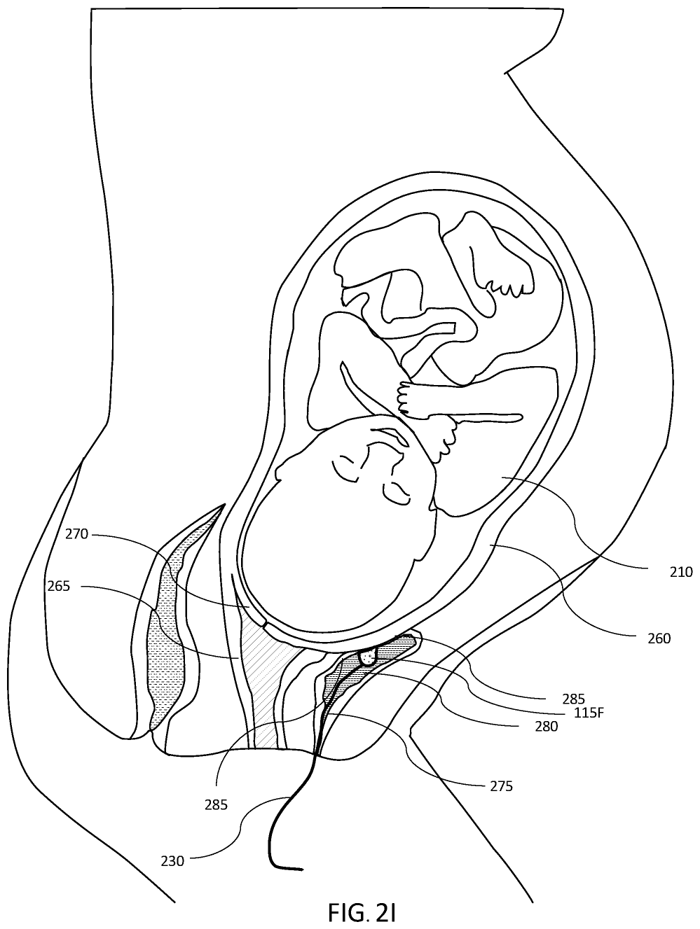

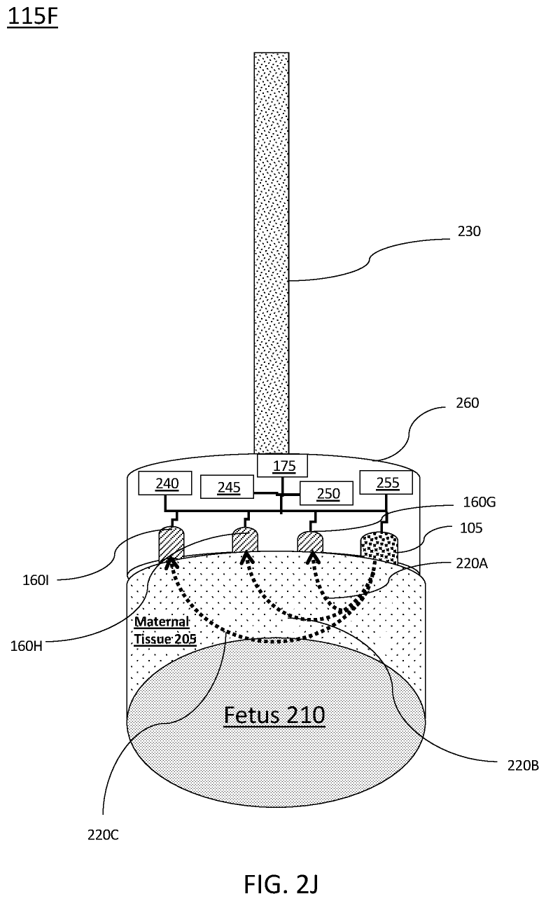

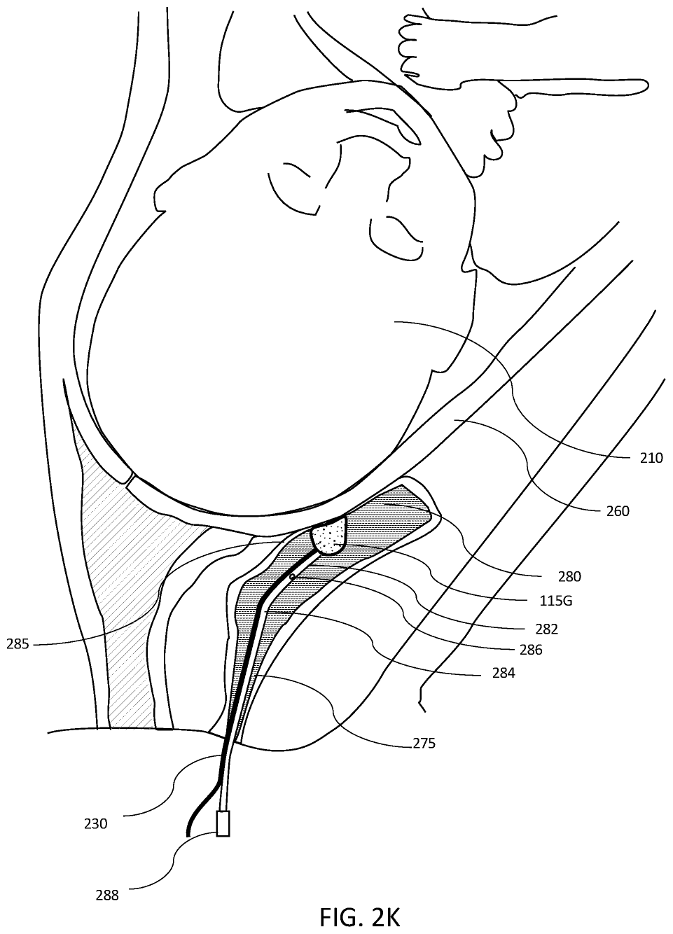

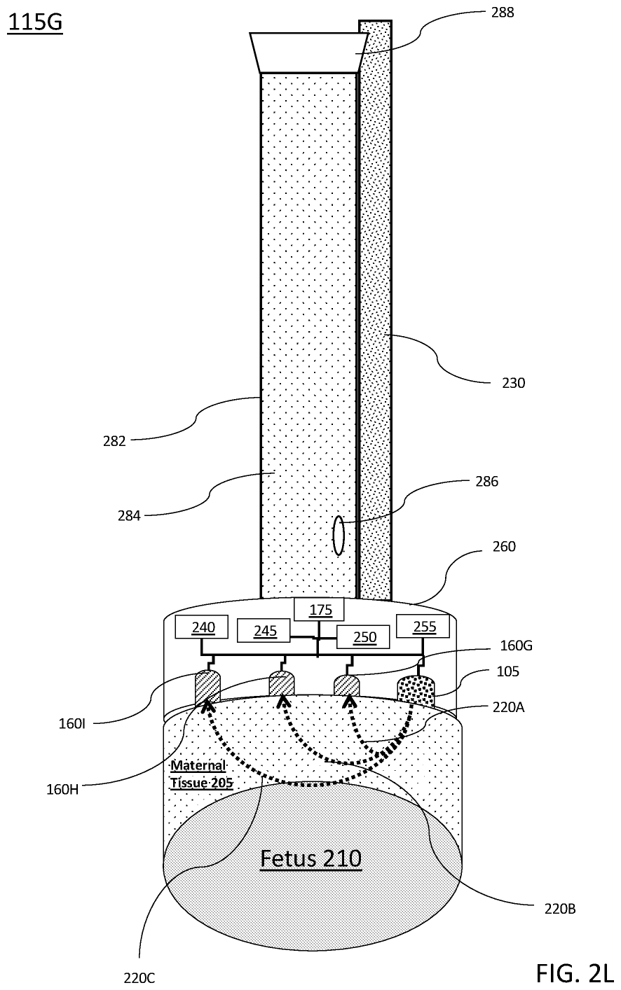

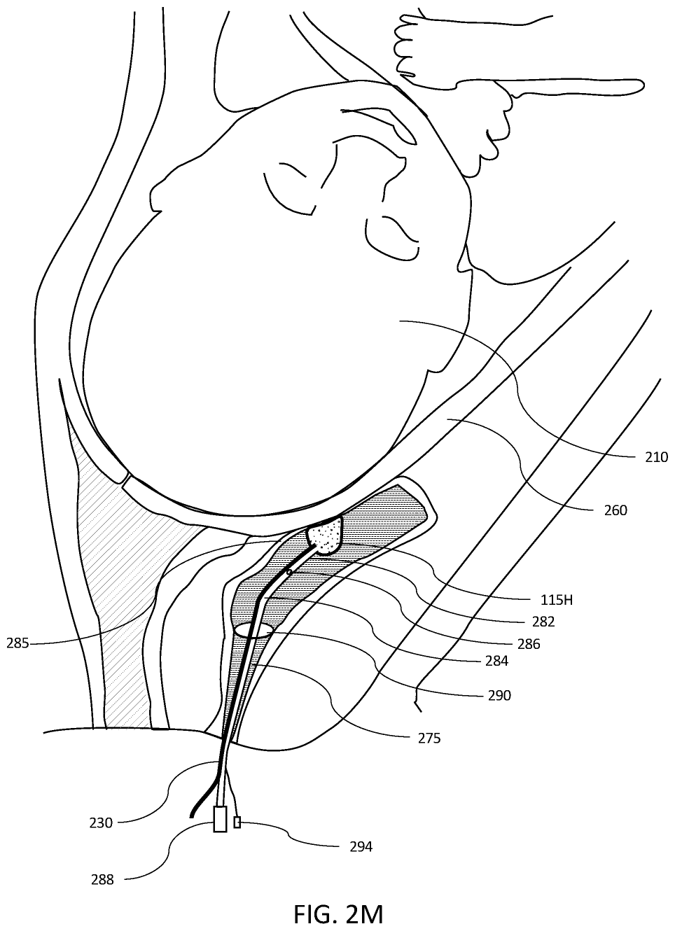

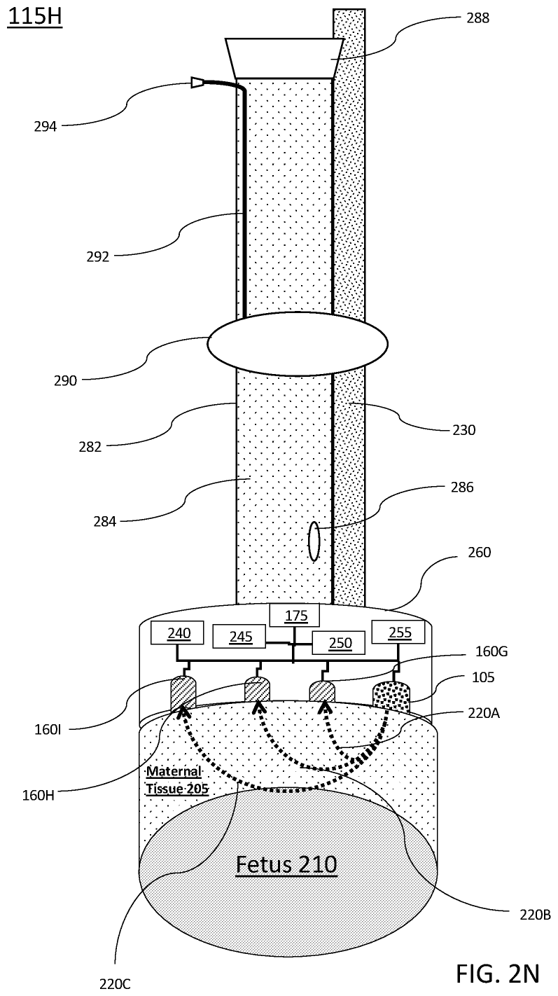

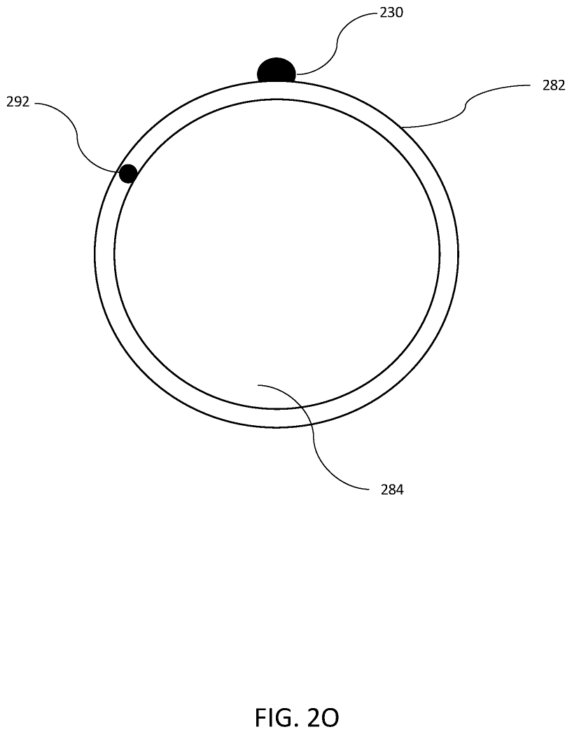

FIG. 2A is a diagram illustrating a cross-section view of an abdomen of a pregnant human woman with a fetus 210 positioned within a uterus 260, wherein an exemplary transvaginal and/or transcervical fetal oximetry probe 115D, which may also be referred to herein as a transvaginal/transcervical fetal oximetry probe 115D is positioned within her endocervical canal 265 and proximate (i.e., touching) to her cervix 270 and FIG. 2B is a close-up view of exemplary transvaginal/transcervical fetal oximetry probe 115D shown in FIG. 2A where the maternal tissue (including, for example, the cervix, amniotic sack, and/or amniotic fluid) is represented as an abstract shape 205 and the fetus is represented as an abstract shape 210. At times, the transvaginal/transcervical fetal oximetry probe(s) discussed herein may be referred to as “transvaginal probe(s)” for the sake of brevity. Also shown in FIG. 2A is the pregnant mammal's urethra 275, bladder 280 and bladder wall 285.

In some embodiments, transvaginal/transcervical fetal oximetry probe 115D may be configured to reside within the pregnant mammal's endocervical canal for an extended period of time (e.g., hours) during, for example, labor and delivery of the fetus. Additionally, or alternatively, transvaginal/transcervical fetal oximetry probe 115D may be configured to reside within the pregnant mammal's endocervical canal on an as-needed and/or periodic (e.g., inserted into and extracted from the endocervical canal) basis over time during, for example, the labor and delivery process.

Transvaginal/transcervical fetal oximetry probe 115D includes a housing 201 with a handle 220 and a body 215. Body 215 includes one light source 105 and three detectors 160G, 160H, and 160I, each of which have a different position relative to source 105 with first detector 160G being the closest to source 105 and third detector 160I being the furthest away from source 105. In some embodiments, handle 220 may include one or more optional components such as ECG machine 175, a transceiver 240, a processor/memory combination 245, a power supply 250, and/or a port 255. Power supply 250 may be any power supply configured to provide electrical power to one or more components of transvaginal/transcervical fetal oximetry probe 115D. In some embodiments, power supply 250 may be a battery (rechargeable or otherwise). Additionally, or alternatively, power supply may be/include an AC/DC. Port 255 may be configured to, for example, provide power to and/or act as a communications interface for transvaginal/transcervical fetal oximetry probe 115D. Exemplary ports 255 include, but are not limited to USB ports, USB-C ports, ethernet ports and the like. In some instances, port 255 may include two or more ports.

Processor/memory 245 may be communicatively coupled to one or more detectors 160G, 160H, and/or 160I and may be configured to receive one or more detected electronic and/or composite signals therefrom. Processor/memory 245 may also be communicatively coupled to light source 105 and may be configured to provide instructions thereto. Exemplary instructions include, but are not limited to, turning light source 105 on/off, a duration of time to project light, light modulation instructions, and/or what type (e.g., wavelength or set of wavelengths) and/or intensity of light to emit. In some embodiments, processor/memory 245 may be configured to pre-process and/or filter detected electronic signals and/or composite signal received from one or more detectors 160G, 160H, and/or 160I. Exemplary pre-processing includes, but is not limited to, filtering (e.g., bandpass or Kalman filter) and/or noise reduction. One or more operations performed by processor/memory 245 may be executed using one or more sets of instructions stored thereon and/or received via, for example, port 255 and/or transceiver 240. At times, these instructions may be updated via communications received via, for example, port 255 and/or transceiver 240.

Transceiver 240 may be communicatively coupled to processor/memory 245, power supply 250, and/or port 255 and may be configured to communicate composite signals and/or detected electronic signals to one or more communicatively connected devices such as computer 150 and/or receiver 145. Transceiver 240 may also be configured to receive instructions regarding the operation of transvaginal/transcervical fetal oximetry probe 115D and provide these instructions to processor/memory 245. Transceiver 240 may be configured to operate via wired and/or wireless communications.

A position of a detector 160G-160I relative to source 105 may be referred to herein as a source/detector distance. In some examples, detectors 160G-160I may be arranged linearly and may be positioned 1 cm apart from one another so that first detector 160G is positioned 1 cm away from source 105, second detector 160H is positioned 1 cm away from first detector 160G, and third detector 160I is positioned 1 cm away from second detector 160H.

Source 105 may project an optical signal 220 into the pregnant mammal's tissue 205 and a resultant optical signal that has reflected off of the maternal tissue 205 and/or fetus 210, and may be detected by one or more of detector(s) 160G-160I. It is expected that the detectors 160 positioned closer to source 105 may detect a portion of the optical signal that has been incident on the pregnant mammal's tissue 205 but not fetus 210 and, in some embodiments, first detector 160G and/or second detector 160H may be positioned via, for example, setting of a source/detector distance, so that a majority, if not all, of an optical signal 220A and 220B detected by first and second detectors 160G and 160H, respectively, has only been incident of the pregnant mammal's tissue 205 (i.e., is not incident on the fetus). Third detector 160I may detect portions of the optical signal 220C that are incident on the pregnant mammal's tissue 205 and fetus 210 as shown in FIG. 2A. In some cases, third detector 160I may be positioned 3-5 cm away from the light source.

FIG. 2C is a diagram illustrating a cross-section view of a pregnant human woman with an exemplary transvaginal/transcervical fetal oximetry probe 115D positioned within her endocervical canal 265, through an opening in cervix 270 as may be the case when the cervix 270 is sufficiently dilated to allow for passage of transvaginal/transcervical fetal oximetry probe 115D therethrough so that transvaginal/transcervical fetal oximetry probe 115D may be positioned proximate to (in some cases touch) her fetus 210 as may be the case when transvaginal/transcervical fetal oximetry probe 115D passes through the cervix and is placed directly on the fetus. FIG. 2D is a close-up view of exemplary transvaginal/transcervical fetal oximetry probe 115D positioned as shown in FIG. 2C where the fetus is shown as an abstract shape 210. When transvaginal/transcervical fetal oximetry probe 115D is positioned directly next to fetus 210, source 105 may project an optical signal 220 into the fetus 210 and a resultant optical signal may be detected by one or more of detector(s) 160G-160I via, for example the optical signal reflecting off of the fetus 210 and being detected by detector(s) 160G-160H.

FIG. 2E is a diagram illustrating a cross-section view of a pregnant human woman with an exemplary transvaginal and/or transcervical fetal oximetry probe 115E, which may also be referred to herein as a transvaginal/transcervical fetal oximetry probe 115E, positioned within her endocervical canal 265 and proximate to her cervix 270 and FIG. 2F is a close-up view of exemplary transvaginal/transcervical fetal oximetry probe 115E shown in FIG. 2E where the maternal tissue (including, for example, the cervix, amniotic sack, and/or amniotic fluid) is represented as abstract shape 205 and the fetus is represented as abstract shape 210.

Transvaginal/transcervical fetal oximetry probe 115E is similar to transvaginal/transcervical fetal oximetry probe 115D but has a different form factor in that transceiver 240, processor/memory combination 245, power supply 250, and/or port 255 are positioned in body 215 instead of in handle 220 and body 215 is attached to a cord 230. Cord 230 may include one or more wires to convey electricity and/or communications to and/or from body 215 and/or components thereof. In embodiments, cord 230 may be configured to enable the mechanical extraction of transvaginal/transcervical fetal oximetry probe 115E from the pregnant mammal's endocervical canal.

Transvaginal/transcervical fetal oximetry probe 115E may be configured with a small form factor so that it is easily inserted into and extracted from the pregnant mammal's endocervical canal. In some embodiments, transvaginal/transcervical fetal oximetry probe 115E may be configured to reside within the pregnant mammal's endocervical canal for an extended period of time (e.g., hours) during, for example, labor and delivery of the fetus. Additionally, or alternatively, transvaginal/transcervical fetal oximetry probe 115E may be configured to reside within the pregnant mammal's endocervical canal on an as-needed and/or periodic basis (e.g., inserted into and extracted from the endocervical canal) over time during, for example, the labor and delivery process.

FIG. 2G is a diagram illustrating a cross-section view of a pregnant human woman with an exemplary transvaginal/transcervical fetal oximetry probe 115E positioned within her endocervical canal 265 and proximate to her fetus 210 (i.e., transvaginal/transcervical fetal oximetry probe 115E has passed through the cervix and is placed directly on the fetus) and FIG. 2H is a close-up view of exemplary transvaginal/transcervical fetal oximetry probe 115E positioned as shown in FIG. 2G where the fetus is shown as abstract shape 210. When transvaginal/transcervical fetal oximetry probe 115E is positioned directly next to fetus 210, source 105 may project an optical signal 220 into the fetus 210 and a resultant optical signal may be detected by one or more of detector(s) 160G-160I via, for example the optical signal reflecting off of the fetus 210 and being detected by detector(s) 160G-160.

FIG. 2I is a diagram illustrating a cross-section view of a pregnant human woman with an exemplary transurethral fetal oximetry probe 115F that has been inserted into the woman's urethra 275 and positioned within the pregnant mammal's bladder 280 proximate to a portion of the bladder wall 285 closest to the pregnant mammal's uterus 260 and her fetus 210 and FIG. 2J is a diagram illustrating the exemplary transurethral fetal oximetry probe 115F positioned proximate to maternal tissue 205 where the maternal tissue 205 includes, for example, bladder wall 285, uterus 260, amniotic sack, etc.

Transurethral fetal oximetry probe 115F may be similar to and/or include components similar to transvaginal/transcervical fetal oximetry probe 115D and/or transvaginal/transcervical fetal oximetry probe 115E however, transurethral fetal oximetry probe 115F may be configured with a form factor small enough (e.g., a 3-15 mm diameter) to enable transurethral insertion and placement of transurethral fetal oximetry probe 115F within the bladder 280. Transurethral fetal oximetry probe 115F may be configured to project light into the maternal tissue 205 and detect a plurality of optical signals 220A, 220B, and 220C as shown in FIG. 2J resultant therefrom.

Although transvaginal/transcervical fetal oximetry probe 115D and 115E and transurethral fetal oximetry probe 115F are shown to include 3 detectors, it will be understood by those of skill in the art that transcervical fetal oximetry probe 115D and 115E and/or transurethral fetal oximetry probe 115F may include any appropriate number (e.g., 1, 2, 4, 5, 6) of detectors 160.

On some occasions when, for example, the pregnant mammal undergoes an epidural for analgesia from labor contractions, the pregnant mammal may require urinary catheterization. On these occasions, use of a device that is both a transurethral fetal oximetry probe and a catheter may be desired so that, for example, only one device needs to be inserted into the pregnant mammal's urethra 275 and/or bladder 280 to be positioned on a portion of bladder wall 285 proximate to the pregnant mammal's uterus 260 and fetus 210. The transurethral fetal oximetry probe/catheter combinations disclosed herein may be used to gather data (typically in the form of detected electronic signals that correspond to an optical signal that was incident on the tissue of a pregnant mammal and/or her fetus) and/or execute any of the methods disclosed herein in a similar manner as a stand-alone transurethral fetal oximetry probe without the catheter components.

One exemplary embodiment of a first combined transurethral fetal oximetry probe/catheter 115G positioned within the pregnant mammal's bladder 280 and proximate to a wall of the bladder 285 closest to her fetus is shown in FIG. 2K and FIG. 2L is a diagram illustrating the first combined transurethral fetal oximetry probe/catheter positioned proximate to an approximation of maternal and fetal tissue. The first exemplary transurethral fetal oximetry probe/catheter combination 115G includes all the components of transurethral fetal oximetry probe 115F along with components of an intermittent urinary catheter such as a tube 282 with a lumen therein 284 configured to allow urine passing through an opening 286 positioned in tube 282 to be evacuated from the pregnant mammal's bladder. Tube 282 also includes a coupling 288 configured to facilitate the coupling of tube 282 to, for example, a bag or other receptacle (not shown) for the collection of urine evacuated from the bladder.

FIGS. 2M and 2N provide a different, or second, embodiment of a combined transurethral fetal oximetry probe/catheter 115H where FIG. 2M is a diagram illustrating a cross-section view of a pregnant human woman with transurethral fetal oximetry probe/catheter combination 115H positioned within the pregnant mammal's bladder 280 and proximate to a wall of the bladder 285 closest to her fetus. Second exemplary transurethral fetal oximetry probe/catheter combination 115H includes all the components of transurethral fetal oximetry probe 115F along with components of an indwelling urinary catheter such as tube 282 with lumen therein 284 configured to allow urine passing through an opening 286 positioned in tube 282 to be evacuated from the pregnant mammal's bladder. Tube 282 also includes coupling 288 and an inflatable balloon 290 that may be used to secure placement of transurethral fetal oximetry probe/catheter combination 115H within the bladder of the pregnant mammal as shown in FIG. 2N. Inflatable balloon 290 may be inflated following placement in bladder 280 and deflated for extraction from bladder 280/urethra 275 via an air/gas tube 292 which is configured with a pump coupling 294 configured to couple to an air/gas pump (not shown) that pumps air/gas into and out of a lumen within air/gas tube 292 for the respective inflation/deflation of inflatable balloon 290.

FIG. 2O is a diagram of a cross section of the second exemplary transurethral fetal oximetry probe/catheter combination that shows tube 282, lumen 284, and air/gas tube 292 within a sidewall of tube 282. FIG. 2O also shows cord 230 positioned on top of tube 282.

Cord 230 of first and/or second exemplary transurethral fetal oximetry probe/catheter combination 115G and 155H may be separate from and/or affixed to tube 282. When cord 230 is affixed to tube 282, the affixation may be accomplished by any appropriate means including, but not limited to, chemical or heat bonding and/or use of a sleeve and/or strap to bind cord 230 to tube 282. Cord 230 may diverge from tube 282 at the end of tube furthest away from the fetal oximetry probe portion of first and/or second exemplary transurethral fetal oximetry probe/catheter combination 115G and 155H so that, for example, cord 230 may be coupled to an external device, such as a computer like computer 150 and/or power source.

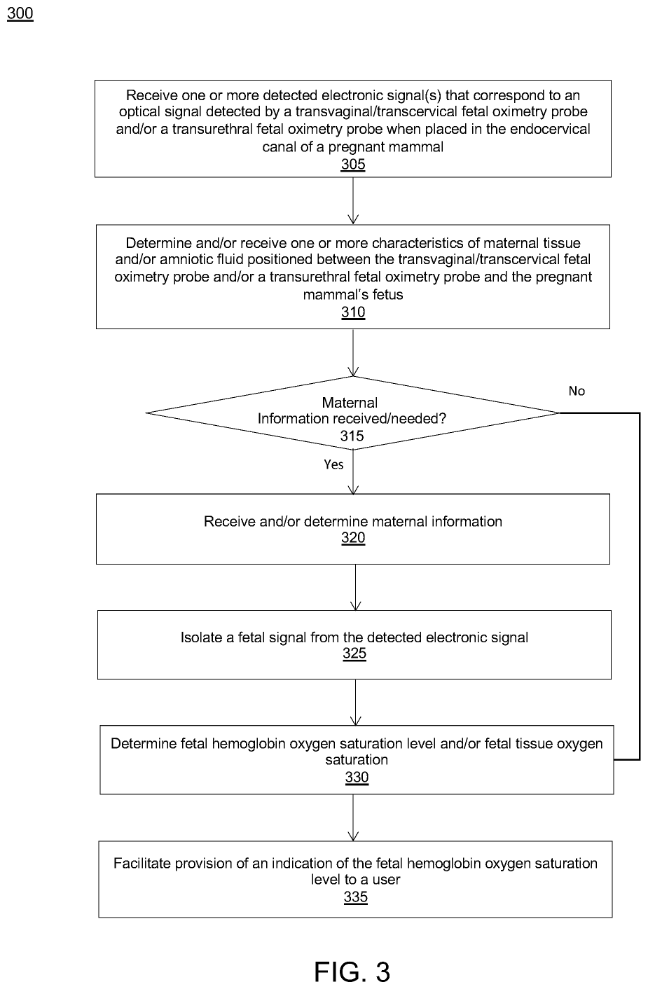

FIG. 3 is a flowchart illustrating a process 300 for performing fetal oximetry and/or fetal pulse oximetry using a transvaginal/transcervical fetal oximetry probe such as transvaginal/transcervical fetal oximetry probe 115D or 115E and/or a transurethral fetal oximetry probe like transurethral fetal oximetry probe 115F. Process 300 may also be executed to determine a level of fetal tissue oxygenation and/or a level of oxygen saturation for fetal hemoglobin. Process 300 may be performed by, for example, system(s) 100, 101, and/or components thereof.

In step 305, one or more detected electronic signal(s) that correspond to one or more respective optical signal(s) of one or more wavelengths detected by a detector like detector 160G, 160H, and/or 160I positioned on/within a transvaginal/transcervical fetal oximetry probe like transvaginal/transcervical fetal oximetry probe 115D when the transvaginal/transcervical fetal oximetry probe is positioned within the endocervical canal and/or cervical canal of a pregnant mammal may be received. Additionally, or alternatively, a detected electronic signal that corresponds to an optical signal of one or more wavelengths detected by a detector like detector 160G, 160H, and/or 160I positioned on/within a transurethral fetal oximetry probe like transurethral fetal oximetry probe 115F may be received in step 305.

The optical signal may be generated by, for example, one or more light sources like light source 105 and may be detected via, for example, reflection and/or back scattering of the projected optical signal from the pregnant mammal's tissue and/or fetus toward the detector. The detector may convert the detected optical signal to an electrical or digital signal which may be communicated to, for example, a computer or processor such as computer 150 that receives the detected electronic signal of step 305. In some embodiments, a detected signal may be received from a different detector like detectors 160G-160I as shown and discussed above with regard to FIGS. 2A-2J.

In the case of multiple detectors in the transvaginal/transcervical and/or transurethral fetal oximetry probe, each detector providing the detected electronic signal received in step 305 may have a different source/detector distance and each detector may be associated with a different detector identifier (e.g., a code). These different detectors may each contribute a different detected electronic signal and/or composite signal, which may include and/or be associated with a respective detector identifier so that, for example, the source/detector distance for a particular detected electronic signal within a group or set of detected electronic signals received in step 305 may be determined.

An exemplary range of wavelengths for the optical signals that correspond to the first detected electronic signals is between 600 and 1000 nm and may be similar to one or more of optical signals 220A-220C as shown in FIGS. 2B, 2D, 2F, 2H, and/or 2J. In some embodiments, the optical signal may be a broadband optical signal (e.g., white light and/or a range of, for example, 10, 15, or 20 wavelengths) and the received detected electronic signal(s) may correspond to an optical signal of a plurality of wavelengths. In some embodiments, the optical signal corresponding to one or more of the detected electronic signals, or a portion thereof, may be of a set, or known, wavelength that may be at an isosbestic point for light directed into human tissue to determine a ratio of oxygenated and de-oxygenated hemoglobin for the human's blood such as 808 nm. Light at this wavelength is reflected from oxygenated and de-oxygenated hemoglobin in the same way.

In step 310, one or more characteristics of the maternal tissue and/or amniotic fluid positioned between the transvaginal/transcervical fetal oximetry probe and the pregnant mammal's fetus may be determined. Exemplary characteristics include, but are not limited to, a fetal depth (i.e., a width of tissue and/or amniotic fluid positioned between the detector of the transvaginal/transcervical fetal oximetry probe and the fetus), a degree of cervical effacement, whether the amniotic sac has ruptured or is positioned between the detector of the transvaginal/transcervical fetal oximetry probe and the fetus, characteristics of the pregnant mammal's cervix (e.g., how dilated it is and/or a thickness of the cervix), characteristics of the pregnant mammal's endocervical canal and/or cervical canal, characteristics of the pregnant mammal's bladder, a thickness of the maternal tissue between the pregnant mammal's bladder wall and the fetus, and/or a composition of the maternal tissue positioned between the pregnant mammal's bladder wall and the fetus. In some embodiments, execution of step 310 may include receiving information from, for example, an ultrasound or MRI image. Additionally, or alternatively, execution of step 310 may include determining and/or receiving a position (e.g., transcervical or not transcervical) of the detector within the endocervical canal, cervical canal, and/or bladder of the pregnant mammal.

In step 315, it may be determined whether maternal information like maternal hemoglobin oxygen saturation level and/or a maternal tissue oxygen saturation level is applicable or is needed to determine fetal tissue and/or hemoglobin oxygenation levels. This determination may be based upon the one or more characteristics determined in step 310. For example, if there is no (or minimal) maternal tissue through which blood flows positioned between the fetus and the transvaginal/transcervical fetal oximetry probe 115D or 115E, then maternal information like maternal hemoglobin oxygen saturation level and/or a maternal tissue oxygen saturation level may not be needed to determine isolate a fetal signal from the detected electronic signal or otherwise determined a fetal hemoglobin and/or tissue oxygenation level and process 300 may proceed to step 330. Alternatively, if there is maternal tissue through which blood flows (e.g., cervical tissue) positioned between the fetus and the transvaginal/transcervical fetal oximetry probe 115D or 115E or transurethral fetal oximetry probe 115G is supplying the detected electronic signals received in step 305, then characteristics of that tissue, or blood flow through that tissue, may be useful in determining a fetal hemoglobin and/or tissue oxygenation level as described below and process 300 may proceed to step 320 wherein maternal information is received and/or determined. The received maternal information may be received from, for example, one or more components of system 100 and may include, for example, a hemoglobin oxygen saturation level, a maternal tissue oxygenation level, and/or a pulse oximetry reading for the pregnant mammal. For example, a pulse oximetry reading and/or hemoglobin oxygen saturation level may be received from a pulse oximetry probe like pulse oximetry probe 130 and/or a maternal pulse oximetry probe like a NIRS adult hemoglobin probe like NIRS adult hemoglobin probe 125. Additionally, or alternatively, an indication of the tissue oxygen saturation level for the pregnant mammal may be received and/or determined in step 320 using, for example, the detected electronic signal received in step 305 and/or a diffuse optical tomography (DOT) instrument and/or may be determined by applying DOT to the detected electronic signals. Additionally, or alternatively, an indication of a hemoglobin and/or tissue oxygen saturation level for the pregnant mammal may be determined using the detected electronic signal received in step 305 and, for example, the Beer-Lambert Law or the modified Beer-Lambert Law in a manner similar that discussed below with regard to Equation 1 and/or Equation 2. In some instances, the pregnant mammal's hemoglobin and/or tissue oxygen saturation level may be used to determine how much light is incident on the fetus as discussed herein and this value (i.e., how much light is incident upon the fetus) may be used to determine the fetal hemoglobin and/or tissue oxygenation via, for example, calculations using Equation 1 and/or 2.

Optionally, in step 325, the detected electronic signal(s) received in step 305 may be processed to isolate a portion thereof that was incident on the fetus. The isolated portion of the detected electronic signal(s) may be referred to herein as a fetal signal(s). In some embodiments, execution of step 325 may resemble execution of step 415 of process 400, discussed below. In embodiments where the maternal information is not needed (in step 315) and/or where the pregnant mammal does not contribute to the detected electronic signal received in 305 (as may be the case when a transvaginal fetal oximetry probe is placed directly on the fetus) and/or does not produce any interference with (e.g., provide any confounding effects) the detected electronic signal received in 305 then, step 325 may be unnecessary because the pregnant mammal's tissue is not confounding the fetal oximetry measurements.

Step 325 may be executed using any appropriate method of isolating a fetal signal from a corresponding detected electronic signal including, but are not limited to, reducing noise in the signal via, for example, application of filtering or amplification techniques, determining a portion of the first detected electronic signal that is contributed by the pregnant mammal and then subtracting, or otherwise removing, that portion of the first detected electronic signal from the received first detected electronic signals and/or receiving information regarding a fetal heart rate and using that information to lock in (via, for example, a lock-in amplifier) on a portion of the received first detected electronic signals generated by the fetus.

Optionally, execution of step 325 may include pre-processing of the detected electronic signal in order to, for example, remove noise from the signal and/or confounding effects of the pregnant mammal's anatomy or physiological signals (e.g., a respiratory signal) from the detected electronic signals. Execution of the pre-processing may include, but is not limited to, application of filtering techniques to the detected electronic signal, application of amplification techniques to the detected electronic signal, utilization of a lock-in amplifier on the detected electronic signal, and so on. In some embodiments, the pre-processing may include application of a filter (e.g., bandpass or Kalman) to the detected electronic signal to reduce noise or hum in the detected electronic signal that may be caused by, for example, electronic noise generated by equipment generating and/or detecting the detected electronic signals and/or environmental equipment (e.g., a ventilator) that may, in some instances, be proximate and/or coupled to the pregnant mammal.

In some embodiments, execution of step 325 may include performing short separation analysis whereby a detected electronic signal corresponding to an optical signal that only passes through maternal tissue (i.e., does not penetrate deeply enough to be incident on the fetus) is used to determined characteristics of the maternal signal that is included in a detected electronic signal that includes both maternal and fetal contributions so that the maternal contributions to the detected electronic signal may be removed therefrom, which may contribute to the isolation of the fetal signal from the detected electronic signals. An exemplary short separation optical signal is optical signals 220A and 220B as shown in FIGS. 2B, 2F, and 2J.

In step 330, a hemoglobin and/or tissue oxygen saturation level for the fetus may be determined using, for example, the modified Beer-Lambert law, which is presented as Equation 1 below, for each wavelength included in the detected electronic signal(s) under study.

$$Δ {μ}_{a} ( λ ) = - \frac{1}{r * DPF ( λ )} \frac{Δ I ( λ )}{{I}_{0}} \qquad Equation 1$$

where:
Δμₐ(λ)=the change in the absorption coefficient for a given wavelength λ over a defined time period;
r=a distance between the light source and detector;
DPF=the differential path length factor for the given wavelength λ;
I₀=the intensity of emitted light of the given wavelength λ (e.g., the number of photons emitted by the light source) and time (t)=0; and
ΔI(λ)=the change in the measured light intensity of detected light (e.g., the number of photons detected by the detector) for the given wavelength λ over the defined time period.
   A value for I₀ for each wavelength of light in an incident optical signal corresponding to the second det under study may be, for example, an intensity of light projected into the pregnant mammal's abdomen and/or an intensity of the light incident on the fetus as may be determined via a process disclosed herein.

In embodiments where a hemoglobin and/or tissue oxygen saturation level of the pregnant mammal is received and/or determined in step 315, the hemoglobin and/or tissue oxygen saturation level may be used to determine how much, or an intensity of, light emitted by a light source that is directed into the abdomen of the pregnant mammal is absorbed by maternal tissue or hemoglobin. A correlation between the hemoglobin and/or tissue oxygen saturation level of the pregnant mammal and how much of the incident light she may absorb for each wavelength of light may be known and/or empirically determined and these correlations may be stored in, for example, a look up table of a database like database 170 such that when a hemoglobin and/or tissue oxygen saturation level for a pregnant mammal is received and/or determined in step 315, it may be used to look up a corresponding level of light absorption (e.g., a percentage or ratio) for the pregnant mammal. This value (the level of light absorption for the pregnant mammal) may then be applied (e.g., subtracted or multiplied) to an initial intensity of a light source when it is projecting light into the pregnant mammal's abdomen to determine the initial intensity of light incident on the fetus (I₀). ΔI(λ) may be the change in the measured intensity of light incident on the fetus (I₀) at wavelength λ and an intensity of a detected fetal signal for light of wavelength λ.

Once the absorption coefficient is determined via Equation 1, an indication of fetal hemoglobin oxygen saturation may be determined via, for example, calculations using Equation 2, provided below:

Δμa(λ)=ΔcHbO*εHbO(λ)+ΔcHb*εHb(λ) Equation 2

where:
Δμₐ(λ)=the change in the absorption coefficient for a given wavelength λ over a defined time period;
Δc_{HbO}=a change in the concentration of oxygenated hemoglobin (HbO) over the defined time period;
Δc_{Hb}=a change in the concentration of deoxygenated hemoglobin (Hb) over the defined time period;
ε_{HbO}(λ)=the extinction coefficient for oxygenated hemoglobin (HbO) for the given wavelength; and
ε_{Hb}(λ)=the extinction coefficient for deoxygenated hemoglobin (Hb) for the given wavelength.

Equation 1 may be solved for two or more wavelength pairs by inputting the change in intensity I, as a function of wavelength λ. From this, changes in absorption coefficients, Δμₐ, may be determined using Equation 2 by inputting known extinction coefficients, εHbO(λ) and εHb(λ) for a particular wavelength, which may be looked up in, for example, a look-up table stored on, for example, computer 150. The wavelength pairs used to perform the calculations of Equation 2 may be any pair of wavelengths included in the spectrum of wavelengths of the optical signal incident upon the pregnant mammal's abdomen. In some embodiments, the calculation of Equation 2 may be performed many times (e.g., 10s, 100s, or 1000s), in different combinations of wavelengths, in order to arrive at multiple values for ΔcHbO and ΔcHb which may be weighted and/or averaged according to one or more criteria to arrive at robust values (e.g., statistically valid and/or with an acceptable level of confidence and error rate) for ΔcHbO and ΔcHb. Additionally, or alternatively, the calculation of Equation 2 may be performed many times (e.g., 10s, 100s, or 1000s), to fit a plurality of wavelengths at the same time to the equation.

The values for ΔcHbO and ΔcHb generated via Equation 2 are relative values, not absolute values, for the concentrations of oxygenated and deoxygenated hemoglobin in the fetus's blood, which may be useful in monitoring changes in the fetal hemoglobin oxygen saturation levels of the fetus over time. In some embodiments, the determination of step 330 may also include determining an overall oxygen saturation for the fetus's hemoglobin by determining a ratio of the change in concentration of oxygenated hemoglobin to the change in concentration of total hemoglobin, which may be the sum of oxygenated and deoxygenated hemoglobin.

Once the fetal hemoglobin oxygen saturation level is determined in step 330, provision of an indication of same to a user may be facilitated by, for example, display on a display device like display device 155 (step 335).

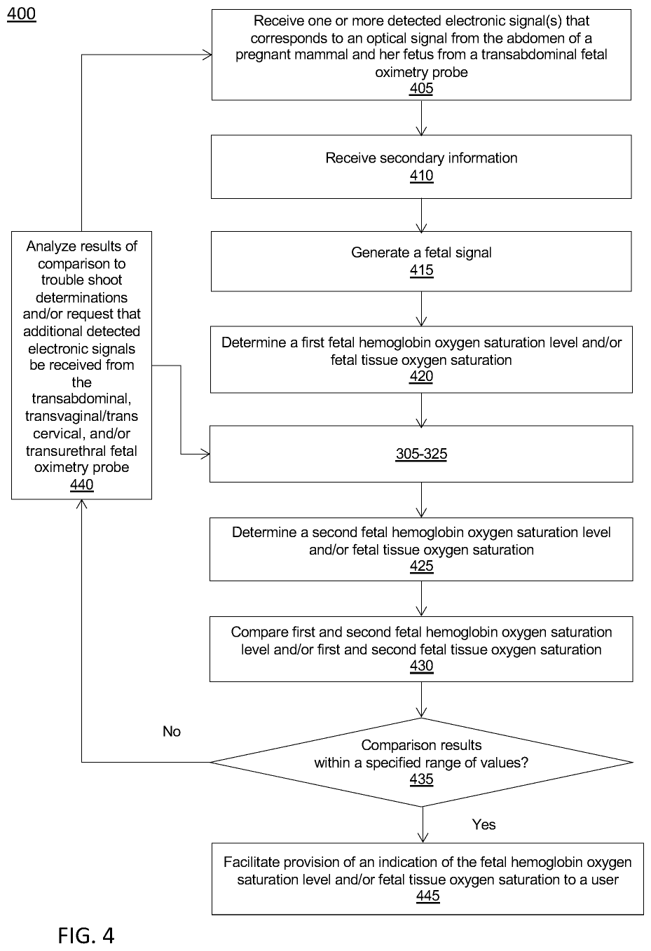

FIG. 4 is a flowchart illustrating a process 400 for performing fetal oximetry and/or fetal pulse oximetry using both a transabdominal fetal oximetry probe and a transvaginal/transcervical fetal oximetry probe, such as transvaginal/transcervical fetal oximetry probe 115D or 115E and/or a transurethral fetal oximetry probe like transurethral fetal oximetry probe 115F. Process 400 may also be executed to determine a level of fetal tissue oxygenation and/or a level of oxygen saturation for fetal hemoglobin. Process 400 may be performed by, for example, system(s) 100, 101, and/or components thereof.

Initially, a detected electronic signal that corresponds to an optical signal exiting from the abdomen of a pregnant mammal and a fetus contained therein may be received (step 405) by, for example, a computer or processor such as computer 150. The detected electronic signal may be received from a transabdominal fetal oximetry probe like transabdominal fetal oximetry probes 115C, 115D, 115E, and/or 115F. The optical signal may correspond to an optical signal of one or more wavelengths projected into the pregnant mammal's abdomen by, for example, one or more light sources like light source 105 and exiting the maternal abdomen via, for example, reflection, back scattering, and/or transmission. In some embodiments, the optical signal may be a broadband optical signal (e.g., white light and/or a range of, for example, 10, 15, or 20 wavelengths) and the received detected signal may correspond to an optical signal of a plurality of wavelengths. The optical signal exiting from the pregnant mammal's abdomen may be detected by a detector like detector 160, 160A, 160B, 160C, 160D, 160E, 160F, 160G, 160H, and/or 160I configured to convert an optical signal (in some cases a single photon) into an electronic signal, which is the detected electronic signal. At times, the detected electronic signal may include an intensity magnitude for different wavelengths of light that may correspond to the optical signal. The detector may then directly and/or indirectly communicate the detected electronic signal to a processor as may be housed in a computer such as computer 150.

The optical signal(s) that correspond to the detected electronic signal(s) may include one or more wavelengths of light generated by, for example, a light source like light source 105 and may be, for example, one or more monochromatic light source(s), one or more broadband light sources. In some embodiments, the optical signal(s) may be filtered and/or polarized. An exemplary range of wavelengths for the optical signal(s) is between 600 and 1000 nm.

Optionally, secondary information may be received in step 410. Exemplary secondary information includes, but is not limited to, a fetal heart rate, a maternal heart rate, a maternal pulse signal, a respiratory signal for the pregnant mammal, a ventilatory signal for the pregnant mammal, an indication of whether meconium has been detected in the amniotic fluid of the pregnant mammal, a signal indicating uterine tone, a signal indicating a hemoglobin oxygen saturation level of the pregnant mammal, a pulse oximetry signal of the pregnant mammal and combinations thereof. In some embodiments, the respiratory signal may be received from a ventilation device providing air, oxygen, and/or other gasses to the pregnant mammal. Often times, this delivery of air, oxygen, and/or other gasses occurs with a periodic frequency (e.g., every 5 or 10 seconds) and this periodic frequency and optionally along with when, in time, the ventilation is delivered to the pregnant mammal (e.g., time=0 seconds, 5 seconds, 10 seconds, etc.) and this periodic frequency may be a secondary signal. When the secondary information is a fetal heart rate signal, the fetal heart rate signal may be received from, for example, Doppler/ultrasound probe 135 and/or an ECG device like ECG 175. When the secondary information is a maternal heart rate signal, the maternal heart rate signal may be received from, for example, pulse oximetry probe 130, NIRS adult hemoglobin probe 125, and/or a blood pressure sensing device.

In step 415, a portion of the detected electronic signal received in step 405 that corresponds to light that was incident on the fetus may be isolated from the detected electronic signal thereby generating a fetal signal. Step 415 may be executed by, for example, using the secondary information to detect a portion of the detected electronic signal contributed by the fetus and/or remove a portion of the detected electronic signal that is contributed by the pregnant mammal. For example, in some instances, execution of step 415 involves using the secondary information (e.g., respiratory signal for the pregnant mammal, fetal heart rate signal, and/or maternal heart rate signal) to isolate, amplify, and/or extract, a portion of the received detected electronic signal such as the portion of the signal contributed by the fetus.

In some embodiments, execution of step 415 may include execution of one or more procedures to, for example, reduce the signal-to-noise ratio or amplify a portion of the detected electronic signal corresponding to light that was incident upon the fetus. These processes include, but are not limited to, application of filters, subtraction of a known noise component, multiplication of two signals, normalization, and removal of a maternal respiratory signal. In some instances, execution of step 415 may include processing the detected electronic signal with a lock-in amplifier to amplify a preferred portion of the detected electronic signal and/or reduce noise in the detected electronic signal. The preferred portion of the signal may, in some instances, correspond to known quantities (e.g., wavelength or frequency) of the light incident on the pregnant mammal's abdomen.

In some embodiments, execution of step 415 to generate the fetal signal may include filtering the detected electronic signal using, for example, the fetal heart rate signal, the maternal heart rate signal, and/or the secondary signal. In one example, a fetal heart rate signal may be received in step 410 and correlated with the detected electronic signal in step 405. Then, a filter (e.g., bandpass and/or Kalman) that captures a range of frequencies that may correspond to, or approximate (e.g., +/−5, 10, 15, or 20%), the fetal heart rate may be applied to the detected electronic signal so that all frequencies included in the detected electronic signal that do not correspond to the fetal heart rate (or an approximation thereof) are removed from the detected electronic signal. For example, if a fetus's heart rate is 3 Hz, then the filter may be set to filter out portions of the signal above 5 Hz and below 1 Hz. In another example, if a fetus's heart rate is 3 Hz, then the filter may be set to filter out portions of the signal above 10 Hz and below 2 Hz. In another example, if a fetus's heart rate is 3 Hz, then the filter may be set to filter out portions of the signal above 3.8 Hz and below 2.2 Hz.

Additionally, or alternatively, in another example, a maternal heart rate signal may be received in step 410 and correlated with the detected electronic signal in step 415. Then, a filter that captures a range of frequencies that may correspond to, or approximate (e.g., +/−10%, 15%, or 20%), the maternal heart rate frequency may be applied to the detected electronic signal so that all frequencies included in the detected electronic signal that correspond to the maternal heart rate (or an approximation thereof) are removed from the detected electronic signal.

Additionally, or alternatively, in another example, a secondary signal in the form of a maternal respiratory and/or ventilatory signal may be received in step 410 and correlated with the detected electronic signal in step 405. Then, a filter that captures a range of frequencies that may correspond to, or approximate (e.g., +/−5, 10, 15, or 20%), the maternal respiratory and/or ventilatory frequency/signal may be applied to the detected electronic signal so that all frequencies included in the detected electronic signal that correspond to the maternal respiratory and/or ventilatory rate are removed from the detected electronic signal.

In some embodiments, the range of frequencies filtered out from the detected electronic signal may be responsive to how dynamic, or irregular, the fetal heart rate, maternal heart rate, and/or secondary signal is so that, for example, the full (or approximately full) range of fetal signal is isolated and/or the full (or approximately full) range of the maternal signal is removed from the detected electronic signal. For example, if over the course of, for example, a 60 second interval the fetal heart rate, maternal heart rate, and/or secondary signal changes little (e.g., +/−2-15%), the then the band of frequencies filter for may be relatively narrow for that 60 second interval. Alternatively, in another example, if over the course of, for example, a 60 second interval the fetal heart rate, maternal heart rate, and/or secondary signal changes more substantially (+/−3-50%), the then the band of frequencies filter for may be relatively wider for that 60 second interval.

Optionally, execution of step 415 may include pre-processing the detected electronic signal in order to, for example, remove noise from the signal and/or confounding effects of the pregnant mammal's anatomy or physiological signals on the first and/or second detected electronic signals. Execution of the pre-processing may include, but is not limited to, application of filtering techniques to the detected electronic signal, application of amplification techniques to the detected electronic signal, utilization of a lock-in amplifier on the detected electronic signal, and so on. In some embodiments, execution of step 415 may include application of a filter (e.g., bandpass or Kalman) to the detected electronic signal, the filtering may reduce noise or hum in the detected electronic signal that may be caused by, for example, electronic noise generated by equipment generating and/or detecting the detected electronic signal and/or environmental equipment that may, in some instances, be coupled to the pregnant mammal. In some instances, this processing may include analysis of the detected electronic signals using information about the pregnant mammal's tissue and/or layers of the pregnant mammal's tissue that may be based upon, for example, ultrasound and/or MRI images, short separation analysis of the pregnant mammal, and/or double short separation analysis of the pregnant mammal to determine optical features and/or oxygenation of the maternal tissue and/or blood. Additionally, or alternatively, the detected electronic signal may be generated using diffuse optical tomography, frequency-domain spectroscopy, and/or time-domain diffuse correlation spectroscopy and use of these techniques may assist with the processing of the detected electronic signal.

In some embodiments, execution of step 415 may include correlating and/or synchronizing the fetal heart rate signal, maternal heart rate signal, and/or secondary signal (when received) with one or more the detected electronic signal(s). In some embodiments the received detected electronic signal and the secondary information may be timestamped with, for example, a baseline starting time (e.g., a date, time, etc. which may be associated with an absolute time (e.g., chronological time) and/or a simultaneous starting point of taking a measurement (e.g., time=0) resulting in the respective received detected electronic, maternal heart rate, fetal heart rate, and/or secondary signal. This timestamping may aid with the synchronization and/or correlation of the detected electronic signals with the secondary information. In some embodiments, the timestamping may take the form of, for example, an electrical ground, an optical signal introduced into an incident optical signal, and/or an acoustic signal that is introduced into the two or more of the received detected electronic, fetal heart rate, maternal heart rate, and/or secondary signals. In one example, an electrical ground, or other interruption (e.g., an intentionally introduced burst of optical noise, acoustic noise, and/or control signal) in the operation of a device that is measuring and/or providing the received detected electronic signals, fetal heart rate signal, maternal heart rate signal, and/or secondary signal may operate as a synchronizing timestamp. This timestamp may serve to provide a synchronized point in time for signals recorded by different devices which may operate on different time scales. This synchronization may assist with alignment of two or more signals so that, for example, a heartbeat provided by maternal heart rate signal may be aligned with a simultaneously generated portion of the detected electronic signal so that, in embodiments where the maternal heart rate is used to isolate the fetal signal from the detected electronic signal, the correct portion of the detected electronic signal is aligned with the proper maternal rate signal. The signals may be timestamped by, for example, timestamping device 185.

The fetal signal may then be analyzed to determine a first fetal hemoglobin oxygen saturation level and/or a tissue oxygen saturation level (step 420) by, for example, application of the Beer-Lambert Law to the fetal signal, application of the modified Beer-Lambert Law (see e.g., Equations 1 and 2 provided herein) to the fetal signal, and/or correlating a component (e.g., intensity, wavelength of light, etc.) of the fetal signal with a known value corresponding fetal hemoglobin oxygen saturation level value, which may, in some instances, be experimentally determined and/or provided via, for example, execution of process 400 or portions thereof. In some embodiments, execution of step 420 may be similar to execution of step 330.

Next, steps 305-325 may be performed and a second fetal hemoglobin oxygen saturation level and/or a tissue oxygen saturation level may be determined (step 425). Step 425 may be executed in a manner similar to the execution of step 330. Then, the first and second fetal hemoglobin oxygen saturation levels and/or tissue oxygen saturation levels may be compared with one another in order to determine one or more differences therebetween (step 430). Then, in step 435, it may be determined whether the comparison results are within a specified range of values. Execution of step 435 may include, for example, determining whether the determined first and second fetal hemoglobin and/or tissue oxygen saturation values fall within a standard of deviation (e.g., +/−1%, 3%, 5%, or 10%) or acceptable range of error when compared with one another. When the first and second fetal hemoglobin and/or tissue oxygen saturation values are not within a specified range of values (e.g., are too different from one another), the results of the comparison may be analyzed to, for example, detect errors, determine a source of errors, or otherwise trouble shoot the determinations of the first and second fetal hemoglobin and/or tissue oxygen saturation values (step 440). Additionally, or alternatively, execution of step 440 may include requesting and/or initiating a repeated execution of step(s) 405-420 and/or the combination of 305-325, 425 and 430. When the first and second fetal hemoglobin and/or tissue oxygen saturation values are within a specified range of values (i.e., not too different from one another), an indication of the fetal hemoglobin and/or tissue oxygen saturation level may be communicated to a display device like display device 155 for display or provision to a user (step 445).

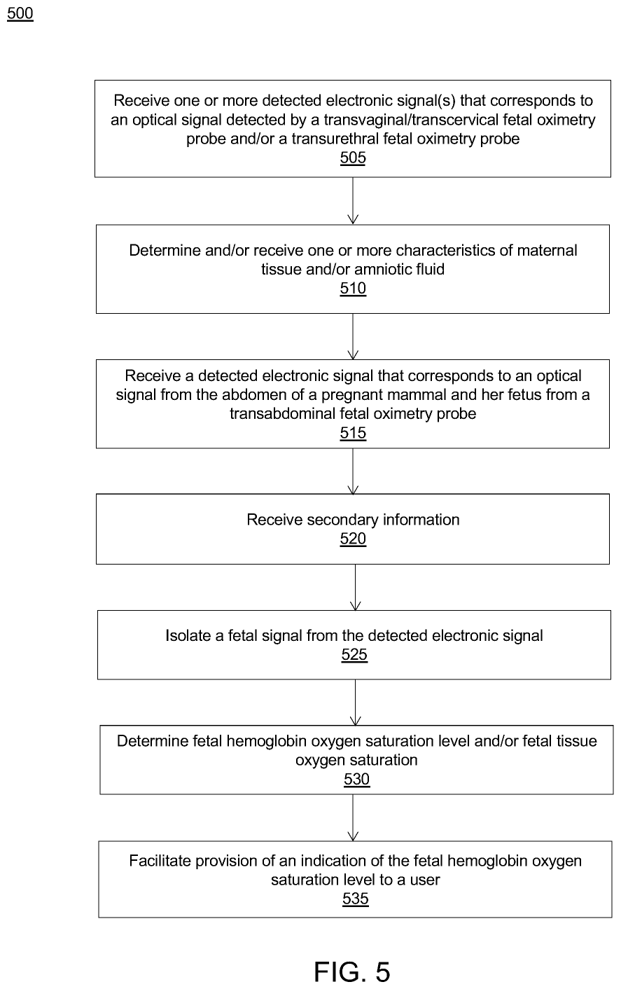

FIG. 5 is a flowchart illustrating a process 500 for performing fetal oximetry and/or fetal pulse oximetry using both a transabdominal fetal oximetry probe and a transvaginal/transcervical fetal oximetry probe, such as transvaginal/transcervical fetal oximetry probe 115D, 115E and/or a transurethral fetal oximetry probe like transurethral fetal oximetry probe 115F. Process 500 may also be executed to determine a level of fetal tissue oxygenation and/or a level of oxygen saturation for fetal hemoglobin. Process 500 may be performed by, for example, system 100, system 101, and/or components thereof.

In step 505, a detected electronic signal that corresponds to an optical signal of one or more wavelengths by a detector like detector 160G, 160H, and/or 160I positioned on/within a transvaginal/transcervical fetal oximetry probe like transvaginal/transcervical fetal oximetry probe 115D when the transvaginal/transcervical fetal oximetry probe is positioned within the endocervical canal and/or cervical canal of a pregnant mammal. Execution of step 505 may be similar to the execution of step 305 described above. The detected electronic signal may by analyzed to determine one or more characteristics of the pregnant mammal's tissue and/or amniotic fluid surrounding the fetus (step 510). Exemplary maternal characteristics include, but are not limited to, a dimension (e.g., composition or width) of maternal tissue, a fetal depth, a degree of scattering caused by the maternal tissue, a degree of light absorbed by the maternal tissue, a maternal tissue oxygenation level, a maternal hemoglobin oxygenation level, a composition of the amniotic fluid (e.g., does it contain meconium), and/or a volume or depth of amniotic fluid positioned between the probe and the fetus.

Next, one or more detected electronic signal(s) that correspond to an optical signal exiting from the abdomen of a pregnant mammal and a fetus contained therein may be received (step 515) by, for example, a computer or processor such as computer 150. In some embodiments, execution of step 515 may be similar to execution of step(s) 305 and/or 405. Optionally, in step 520, secondary information may be received. In some embodiments, execution of step 520 may be similar to execution of step(s) 320 and/or 410. In step 525, the fetal signal may be isolated from the detected electronic signal of step 515. In some embodiments, execution of step 525 may be similar to execution of step(s) 325 and/or 415. Next, the fetal signal may be analyzed using the one or more characteristics determined in step 510 to determine a fetal hemoglobin oxygen saturation level and/or a tissue oxygen saturation level (step 530). Execution of step 530 may resemble execution of step 420 except that when step 530 is executed, the determination of step 510 is taken into account during the execution of step 530. Additionally, or alternatively, in some embodiments, execution of step 530 may incorporate execution of one or more steps of processes 300 and/or 400 and, in particular, may resemble execution of steps 330, 420, and/or 425. Then, an indication of the fetal hemoglobin and/or tissue oxygen saturation level may be communicated to a display device like display device 155 for display or provision to a user (step 535).

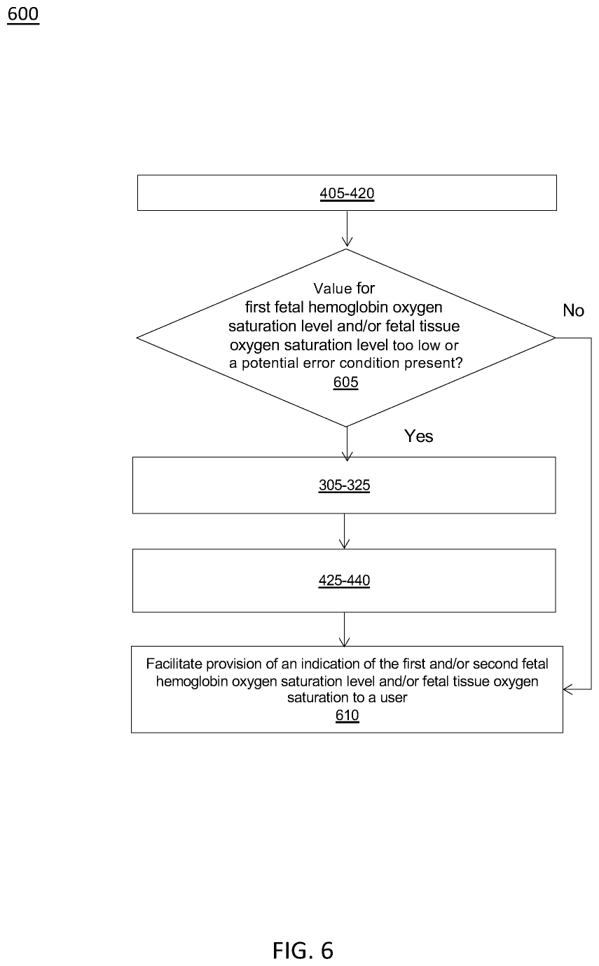

FIG. 6 is a flowchart illustrating a process 600 for verifying a determination of fetal hemoglobin and/or tissue oxygen saturation made by a transabdominal fetal oximetry probe using a transvaginal/transcervical fetal oximetry probe, such as transvaginal/transcervical fetal oximetry probe 115D or 115E and/or a transurethral fetal oximetry probe like transurethral fetal oximetry probe 115F. Process 600 may be performed by, for example, system 100, system 101, and/or components thereof.

Initially, steps 405-420 may be performed to determine a fetal hemoglobin and/or tissue oxygen saturation level using an optical signal emitted by the abdomen of a pregnant mammal that has been detected by a transabdominal fetal oximetry probe. In step 605, it may be determined whether a value for the fetal hemoglobin and/or tissue oxygen saturation is too low (i.e., an indication that the fetus may be in distress) and/or if there is an indication of a potential error condition present in the determination of the fetal hemoglobin and/or tissue oxygen saturation (e.g., insufficient data to make a determination with a required level of confidence, a value that is above a threshold that represents a “normal” value for a fetal hemoglobin and/or tissue oxygen saturation, etc.).

When the value for the fetal hemoglobin and/or tissue oxygen saturation is too low and/or if there is an indication of a potential error condition present in the determination of the fetal hemoglobin and/or tissue oxygen saturation (step 605), steps 305-325 and 425-440 may be executed to determine a second fetal hemoglobin and/or tissue oxygen saturation level in order to, for example, verify or validate the first fetal hemoglobin and/or tissue oxygen saturation level determined in step 420. When the value for the fetal hemoglobin and/or tissue oxygen saturation is not too low and/or if there is not an indication of a potential error condition present in the determination of the fetal hemoglobin and/or tissue oxygen saturation (step 605), or following execution of step 440 in process 600, an indication of the first and/or second fetal hemoglobin and/or tissue oxygen saturation level may be communicated to a user via, for example, a display device (step 610).

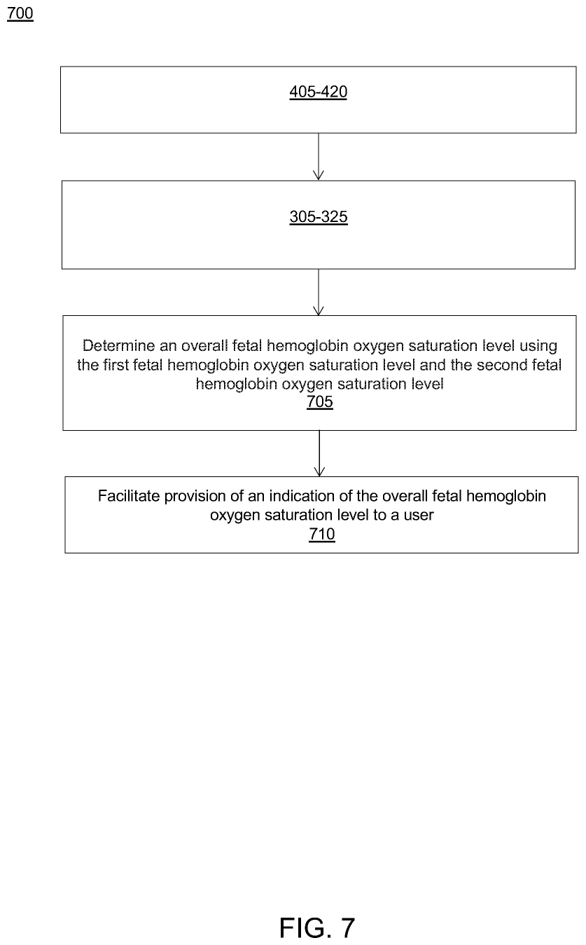

FIG. 7 is a flowchart illustrating a process 700 for determining an overall fetal hemoglobin using a detected electronic signal from a transabdominal fetal oximetry probe and a detected electronic signal from a transvaginal/transcervical fetal oximetry probe, such as transvaginal/transcervical fetal oximetry probe 115D and/or 115E and/or a transurethral fetal oximetry probe like transurethral fetal oximetry probe 115F. Process 700 may be performed by, for example, system 100, system 101, and/or components thereof.

Initially, steps 405-420 of process 400 and steps 305-325 of process 300 may be performed in either order (e.g., steps 405-420 first and then steps 305-325; or vise versa). Then, an overall fetal hemoglobin oxygen saturation level may be determined using the first and second fetal hemoglobin oxygen saturation levels (step 705). Step 705 may be performed by, for example, averaging the first and second fetal hemoglobin oxygen saturation levels together, adding the first and second fetal hemoglobin oxygen saturation levels together, calculating a time-weighted average of the first and second fetal hemoglobin oxygen saturation levels, and/or calculating a weighted average fetal hemoglobin oxygen saturation level using the first and second fetal hemoglobin oxygen saturation levels. In the embodiment where a weighted average is used, the first and second fetal hemoglobin oxygen saturation level may each be assigned a confidence level, or weight, based on, for example, an accuracy level of the fetal oximetry probe providing the detected electronic signals and/or a level of noise in the first and/or second fetal hemoglobin oxygen saturation level. In some embodiments, the weight, or confidence level, assigned to the first and second fetal hemoglobin oxygen saturation levels may be static, or constant, over time and may be based on empirically derived factors. Additionally, or alternatively, the weight, or confidence level, assigned to the first and second fetal hemoglobin oxygen saturation levels may be dynamic, or change, over time based on, for example one or more factors (that may be determined in situ) including, but not limited to, noise, maternal physiology, received maternal information, secondary information, and so on.

Hence, systems, devices, and methods for determining fetal oxygen level have been herein disclosed. In some embodiments, use of the systems, devices, and methods described herein may be particularly useful during the labor and delivery of the fetus (e.g., during the first and/or second stage of labor) because it is difficult to assess fetal health during the labor and delivery process.

More particularly, systems, devices, and methods for using fetal depth to select a calibration factor for calculating fetal hemoglobin oxygen saturation and/or fetal tissue oxygen saturation have been herein disclosed. In addition, systems, devices, and methods for determining fetal depth by analyzing an intensity of detected electronic signals as a function of source/detector distance have been herein disclosed. In addition, systems, devices, and methods for determining fetal depth by determining a time of flight for detected photons have been herein disclosed. In addition, systems, devices, and methods for using maternal hemoglobin oxygen saturation to determine how much light reaches the fetus (i.e., light intensity for light incident on the fetus) and then using the intensity of light incident of light incident on the fetus to analyze a detected electronic signal to determine fetal hemoglobin oxygen saturation and/or fetal tissue oxygen saturation have been herein disclosed.

We claim:

1. A transvaginal fetal oximetry probe comprising:
a housing configured to house a light source and a detector, the housing being sized and shaped to be inserted into an endocervical canal of a pregnant mammal and be positioned proximate to a cervix of the pregnant mammal;
the light source configured to project light of a plurality of wavelengths into the endocervical canal of a pregnant mammal to be incident on a fetus within the pregnant mammal's abdomen; and
the detector configured to detect light reflected from the pregnant mammal's tissue and the fetus and convert the detected light into a detected electronic signal that is communicated to an external processor configured to determine a level of fetal hemoglobin oxygen saturation with the detected electronic signal.

2. The transvaginal fetal oximetry probe of claim 1, further comprising:
a cord that extends from the housing and is configured to electrically couple the transvaginal fetal oximetry probe to a power source and communicate a signal from the detector to the external processor.

3. The transvaginal fetal oximetry probe of claim 1 or 2, further comprising:
a processor configured to pre-process the detected electronic signal prior to communication of the detected electronic signal to the external processor.

4. The transvaginal fetal oximetry probe of claim 1, 2, or 3, wherein the detector is a first detector and the detected electronic signal is a first detected electronic signal, the transvaginal fetal oximetry probe further comprising;
a second detector configured to detect light reflected from the pregnant mammal's tissue and the fetus and convert the detected light into a second detected electronic signal.

5. The transvaginal fetal oximetry probe of claim 2, wherein the cord is configured to facilitate extraction of the transvaginal fetal oximetry probe from the pregnant mammal's endocervical canal.

6. The transvaginal fetal oximetry probe of any of claims 1-5, wherein the housing is further configured to be inserted into the endocervical canal, through a cervical opening, and be positioned proximate to the fetus.

7. The transvaginal fetal oximetry probe of any of claims 1-6, wherein the housing further comprises a power supply.

8. The transvaginal fetal oximetry probe of any of claims 1-7, wherein the housing further comprises a transceiver.

9. A transcervical fetal oximetry probe comprising:
a housing configured to house a light source and a detector, the housing being sized and shaped to be inserted into an endocervical canal of a pregnant mammal and be positioned proximate the fetus;
the light source configured to project light of a plurality of wavelengths into the endocervical canal of a pregnant mammal to be incident on a fetus within the pregnant mammal's abdomen;
the detector configured to detect light reflected from the pregnant mammal's tissue and the fetus and convert the detected light into a detected electronic signal that is communicated to an external processor configured to determine a level of fetal hemoglobin oxygen saturation with the detected electronic signal.

10. The transcervical fetal oximetry probe of claim 9, further comprising:
a cord that extends from the housing and is configured to electrically couple the transcervical fetal oximetry probe to a power source and communicate a signal from the detector to the external processor.

11. The transcervical fetal oximetry probe of claim 9 or 10, further comprising:
a processor configured to pre-process the detected electronic signal prior to communication of the detected electronic signal to the external processor.

12. The transcervical fetal oximetry probe of claim 9, 10, or 11, wherein the detector is a first detector and the detected electronic signal is a first detected electronic signal, the transvaginal fetal oximetry probe further comprising;
a second detector configured to detect light reflected from the fetus and convert the detected light into a second detected electronic signal.

13. The transcervical fetal oximetry probe of any of claims 9-12, wherein the cord is configured to facilitate extraction of the transvaginal fetal oximetry probe from the pregnant mammal's endocervical canal.

14. The transcervical fetal oximetry probe of any of claims 9-13, wherein the housing further comprises a power supply.

15. The transcervical fetal oximetry probe of any of claims 9-14, wherein the housing further comprises a transceiver.

16. A transurethral fetal oximetry probe comprising:
a housing configured to house a light source and a detector, the housing being sized and shaped to be inserted into a urethra of a pregnant mammal and be positioned proximate to a wall of a bladder of the pregnant mammal proximate to the fetus;
the light source configured to project light of a plurality of wavelengths into the tissue of the pregnant mammal to be incident on a fetus within the pregnant mammal's uterus;
the detector configured to detect light reflected from the pregnant mammal's tissue and the fetus and convert the detected light into a detected electronic signal that is communicated to an external processor configured to determine a level of fetal hemoglobin oxygen saturation with the detected electronic signal.

17. The transurethral fetal oximetry probe of claim 16, further comprising:
a cord that extends from the housing and is configured to electrically couple the transurethral fetal oximetry probe to a power source and communicate a signal from the detector to the external processor.

18. The transurethral fetal oximetry probe of claim 16 or 17, further comprising;
a processor configured to pre-process the detected electronic signal prior to communication of the detected electronic signal to the external processor.

19. The transurethral fetal oximetry probe of claim 16, 17, or 18 wherein the detector is a first detector and the detected electronic signal is a first electronic signal, the transurethral fetal oximetry probe further comprising;
a second detector configured to detect light reflected from the pregnant mammal's tissue and the fetus and convert the detected light into a second detected electronic signal.

20. The transurethral fetal oximetry probe of claim 17, wherein the cord is configured to facilitate extraction of the transurethral fetal oximetry probe from the pregnant mammal's urethra.

21. The transurethral fetal oximetry probe of any of claims 16-20, wherein the housing further comprises a power supply.

22. The transurethral fetal oximetry probe of any of claims 16-21, wherein the housing further comprises a transceiver.

23. A method comprising:
receiving, by a processor, a first detected electronic signal from a transabdominal fetal oximetry probe;
determining, by the processor, a first fetal hemoglobin oxygen saturation level using the first detected electronic signal;
receiving, by the processor, a second detected electronic signal from a transvaginal fetal oximetry probe;
determining, by the processor, a second fetal hemoglobin oxygen saturation level using the second detected electronic signal;
comparing, by the processor, the first fetal hemoglobin oxygen saturation level to the second fetal hemoglobin oxygen saturation level; and
determining, by the processor, whether the first fetal hemoglobin oxygen saturation level and the second fetal hemoglobin oxygen saturation level are within a specified range of values and, if so, facilitating provision of an indication of the first and second fetal hemoglobin oxygen saturation level to a user.

24. The method of claim 23, wherein the first detected electronic signal is timestamped and the determining of the first fetal hemoglobin oxygen saturation level comprises:
receiving, by the processor, a timestamped maternal heart beat signal;
synchronizing, by the processor, the maternal heart beat signal and the first detected electronic signal using a timestamp of the maternal heart beat signal and a timestamp first detected electronic signal;
isolating, by the processor, a fetal signal from the detected electronic signal by subtracting portions of the first detected electronic signal that correspond to the maternal heart beat signal; and
calculating, by the processor, the first fetal hemoglobin oxygen saturation level using the fetal signal.

25. The method of claim 23 or 24, wherein the second detected electronic signal is timestamped and the determining of the second fetal hemoglobin oxygen saturation level comprises:
receiving, by the processor, a timestamped maternal heart beat signal;
synchronizing, by the processor, the maternal heart beat signal and the second detected electronic signal using a timestamp of the maternal heart beat signal and a timestamp second detected electronic signal;
isolating, by the processor, a fetal signal from the second detected electronic signal by subtracting portions of the second detected electronic signal that correspond to the maternal heart beat signal; and
calculating, by the processor, the second fetal hemoglobin oxygen saturation level using the fetal signal.

26. The method of claim 23, 24, or 25, wherein the first detected electronic signal is timestamped and the determining of the first fetal hemoglobin oxygen saturation level comprises:
receiving, by the processor, a timestamped fetal heart beat signal;
synchronizing, by the processor, the fetal heart beat signal and the first detected electronic signal using a timestamp of the fetal heart beat signal and a timestamp first detected electronic signal;
isolating, by the processor, a fetal signal from the first detected electronic signal by amplifying portions of the first detected electronic signal that correspond to the fetal heart beat signal; and
calculating, by the processor, the first fetal hemoglobin oxygen saturation level using the fetal signal.

27. The method of any of claims 23-26, wherein the second detected electronic signal is timestamped and the determining of the second fetal hemoglobin oxygen saturation level comprises:
receiving, by the processor, a timestamped fetal heart beat signal;
synchronizing, by the processor, the fetal heart beat signal and the second detected electronic signal using a timestamp of the fetal heart beat signal and a timestamp second detected electronic signal;
isolating, by the processor, a fetal signal from the second detected electronic signal by amplifying portions of the second detected electronic signal that correspond to the fetal heart beat signal; and
calculating, by the processor, the second fetal hemoglobin oxygen saturation level using the fetal signal.

28. The method of any of claims 23-28, further comprising:
receiving, by the processor, a characteristic of the pregnant mammal, wherein determining first fetal hemoglobin oxygen saturation level further uses the characteristic of the pregnant mammal.

29. The method of any of claims 23-28, further comprising:
receiving, by the processor, a characteristic of the pregnant mammal, wherein determining second fetal hemoglobin oxygen saturation level further uses the characteristic of the pregnant mammal.

30. The method of claim 28 or 29, wherein the characteristic of the pregnant mammal is one or more of maternal hemoglobin oxygen saturation level, maternal heart rate, thickness of maternal tissue the light passes through, type of maternal tissue the light passes through, maternal blood pressure, and maternal respiratory rate.

31. A method comprising:
receiving, by a processor, a first detected electronic signal from a transabdominal fetal oximetry probe;
determining, by the processor, a first fetal hemoglobin oxygen saturation level using the first detected electronic signal;
receiving, by the processor, a second detected electronic signal from a transvaginal fetal oximetry probe;
determining, by the processor, a second fetal hemoglobin oxygen saturation level using the second detected electronic signal;
determining, by the processor, an overall fetal hemoglobin oxygen saturation level using the first fetal hemoglobin oxygen saturation level and the second fetal hemoglobin oxygen saturation level; and
facilitating, by the processor, provision of an indication of the overall fetal hemoglobin oxygen saturation level to a user.

32. The method of claim 31, wherein the first detected electronic signal is timestamped and the determining of the first fetal hemoglobin oxygen saturation level comprises:
receiving, by the processor, a timestamped maternal heart beat signal;
synchronizing, by the processor, the maternal heart beat signal and the first detected electronic signal using a timestamp of the maternal heart beat signal and a timestamp first detected electronic signal;
isolating, by the processor, a fetal signal from the detected electronic signal by
subtracting portions of the first detected electronic signal that correspond to the maternal heart beat signal; and
calculating, by the processor, the first fetal hemoglobin oxygen saturation level using the fetal signal.

33. The method of claim 31 or 32, wherein the second detected electronic signal is timestamped and the determining of the second fetal hemoglobin oxygen saturation level comprises:
receiving, by the processor, a timestamped maternal heart beat signal;
synchronizing, by the processor, the maternal heart beat signal and the second detected electronic signal using a timestamp of the maternal heart beat signal and a timestamp second detected electronic signal;
isolating, by the processor, a fetal signal from the second detected electronic signal by subtracting portions of the second detected electronic signal that correspond to the maternal heart beat signal; and
calculating, by the processor, the second fetal hemoglobin oxygen saturation level using the fetal signal.

34. The method of claim 31, 32, or 33 wherein the first detected electronic signal is timestamped and the determining of the first fetal hemoglobin oxygen saturation level comprises:
receiving, by the processor, a timestamped fetal heart beat signal;
synchronizing, by the processor, the fetal heart beat signal and the first detected electronic signal using a timestamp of the fetal heart beat signal and a timestamp first detected electronic signal;
isolating, by the processor, a fetal signal from the first detected electronic signal by amplifying portions of the first detected electronic signal that correspond to the fetal heart beat signal; and
calculating, by the processor, the first fetal hemoglobin oxygen saturation level using the fetal signal.

35. The method of any of claims 31-34, wherein the second detected electronic signal is timestamped and the determining of the second fetal hemoglobin oxygen saturation level comprises:
receiving, by the processor, a timestamped fetal heart beat signal;
synchronizing, by the processor, the fetal heart beat signal and the second detected electronic signal using a timestamp of the fetal heart beat signal and a timestamp second detected electronic signal;
isolating, by the processor, a fetal signal from the second detected electronic signal by amplifying portions of the second detected electronic signal that correspond to the fetal heart beat signal; and
calculating, by the processor, the second fetal hemoglobin oxygen saturation level using the fetal signal.

36. The method of claim 35, further comprising:
receiving, by the processor, a characteristic of the pregnant mammal, wherein determining first fetal hemoglobin oxygen saturation level further uses the characteristic of the pregnant mammal.

37. The method of claim 36, further comprising:
receiving, by the processor, a characteristic of the pregnant mammal, wherein determining second fetal hemoglobin oxygen saturation level further uses the characteristic of the pregnant mammal.

38. The method of claim 36 or 37, wherein the characteristic of the pregnant mammal is one or more of maternal hemoglobin oxygen saturation level, maternal heart rate, thickness of maternal tissue the light passes through, type of maternal tissue the light passes through, maternal blood pressure, and maternal respiratory rate.

39. A method comprising:
receiving, by a processor, a first detected electronic signal from a transabdominal fetal oximetry probe;
determining, by the processor, a first fetal hemoglobin oxygen saturation level using the first detected electronic signal;
receiving, by the processor, a second detected electronic signal from a transurethral fetal oximetry probe;
determining, by the processor, a second fetal hemoglobin oxygen saturation level using the second detected electronic signal;
comparing, by the processor, the first fetal hemoglobin oxygen saturation level to the second fetal hemoglobin oxygen saturation level; and
determining, by the processor, whether the first fetal hemoglobin oxygen saturation level and the second fetal hemoglobin oxygen saturation level are within a specified range of values and, if so, facilitating provision of an indication of the first and second fetal hemoglobin oxygen saturation level to a user.

40. A method comprising:
receiving, by a processor, a first detected electronic signal from a transabdominal fetal oximetry probe;
determining, by the processor, a first fetal hemoglobin oxygen saturation level using the first detected electronic signal;
receiving, by the processor, a second detected electronic signal from a transurethral fetal oximetry probe;
determining, by the processor, a second fetal hemoglobin oxygen saturation level using the second detected electronic signal;
determining, by the processor, an overall fetal hemoglobin oxygen saturation level using the first fetal hemoglobin oxygen saturation level and the second fetal hemoglobin oxygen saturation level; and
facilitating, by the processor, provision of an indication of the overall fetal hemoglobin oxygen saturation level to a user.
